(19) United States
(12) Patent Application Publication
(10) Pub. No.: US 2017/0368208 A1
(43) Pub. Date: Dec. 28, 2017

(54) CANCER IMAGING AGENT

(71) Applicant: ENDOCYTE, INC., West Lafayette, IN (US)

(72) Inventor: Le-Cun XU, West Lafayette, IN (US)

(21) Appl. No.: 15/542,230

(22) PCT Filed: Jan. 8, 2016
(86) PCT No.: PCT/US2016/012653

§ 371 Date: Jul. 7, 2017

Related U.S. Application Data
(60) Provisional application No. 62/102,036, filed on Jan. 11, 2015.
(60) Provisional application No. 62/171,670, filed on Jun. 5, 2015.

(51) Int. Cl. A61K 51/04; A61K 51/08; C07K 5/02; C07K 5/09; G01N 30/02; C07F 13/00; G01N 30/90

(52) U.S. Cl. CPC A61K 51/0459; C07K 5/0202; A61K 51/088; A61K 51/0474; C07K 5/0815; C07F 13/00; G01N 30/90; G01N 2030/027

(57) ABSTRACT

The present disclosure relates to imaging agent formulations, methods for preparing imaging agent formulations and methods for using the same. The present disclosure also relates to kits for imaging agent formulations.

### CROSS REFERENCE TO RELATED APPLICATIONS

This application claims priority under 35 U.S.C. §119(e) to U. S. Provisional Application Ser. No. 62/102,036, filed Jan. 11, 2015 and U. S. Provisional Application Ser. No. 62/171,670, filed Jun. 5, 2015, both of which are incorporated herein by reference in their entirety.

### FIELD OF THE INVENTION

The present disclosure relates to imaging agent formulations, methods for preparing imaging agent formulations and methods for using the same. The present disclosure also relates to kits for imaging agent formulations.

### BACKGROUND

The prostate is one of the male reproductive organs found in the pelvis below the urinary bladder. It functions to produce and store seminal fluid which provides nutrients and fluids that are vital for the survival of sperm introduced into the vagina during reproduction. Like many other tissues, the prostate glands are also prone to develop either malignant (cancerous) or benign (non-cancerous) tumors. The American Cancer Society predicted that over 230,000 men would be diagnosed with prostate cancer and over 30,000 men would die from the disease in the year 2005. In fact, prostate cancer is one of the most common male cancers in western societies, and is the second leading form of malignancy among American men. Current treatment methods for prostate cancer include hormonal therapy, radiation therapy, surgery, chemotherapy, photodynamic therapy, and combination therapy. The selection of a treatment generally varies depending on the stage of the cancer. However, many of these treatments affect the quality of life of the patient, especially those men who are diagnosed with prostate cancer over age 50.

Prostate specific membrane antigen (PSMA) is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. PSMA plays a role in the central nervous system, where it metabolizes N-acetyl-aspartyl glutamate (NAAG) into glutamic and N-acetyl aspartic acid. Accordingly, it is also sometimes referred to as an N-acetyl alpha linked acidic dipeptidase (NAALADase). PSMA is also sometimes referred to as a folate hydrolase I (FOLH I) or glutamate carboxypeptidase (GCP II) due to its role in the proximal small intestine where it removes y-linked glutamate from poly-γ-glutamated folate and α-linked glutamate from peptides and small molecules.

PSMA is named largely due to its higher level of expression on prostate cancer cells; however, its particular function on prostate cancer cells remains unresolved. PSMA is over-expressed in the malignant prostate tissues when compared to other organs in the human body such as kidney, proximal small intestine, and salivary glands. Unlike many other membrane-bound proteins, PSMA undergoes rapid internalization into the cell in a similar fashion to cell surface bound receptors like vitamin receptors. PSMA is internalized through clathrin-coated pits and subsequently can either recycle to the cell surface or go to lysosomes. It has been suggested that the dimer and monomer form of PSMA are inter-convertible, though direct evidence of the interconversion is being debated. Even so, only the dimer of PSMA possesses enzymatic activity, and the monomer does not.

Though the activity of PSMA on the cell surface of prostate cancer cells remains under investigation, it has been recognized by the inventors herein that PSMA represents a viable target for the selective and/or specific delivery of biologically active agents, including imaging agents to such prostate cancer cells. One such imaging agent is of the formula I

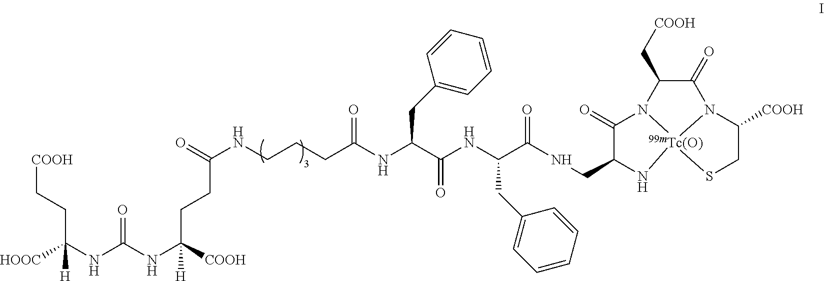

(also referred to herein as ^{99m}Tc-Compound II) as described in WO2009/026177, which is incorporated herein by reference. Compound I has found use as a cancer imaging agent as described in, for example, WO2009/026177. One of skill in the art will recognize that compound (I) can exist as syn- and anti-isomers in reference to the relative position of the Tc═O double bond.

Because imaging agent (I) is of interest in the area of prostate cancer imaging, more efficient procedures for producing imaging agents having higher radioactive purity are desired.

Furthermore, vitamin receptors, such as the high-affinity folate receptor (FR), play an important role in nucleotide biosynthesis and cell division, intracellular activities which occur in both malignant and certain normal cells. The FR is a prime example of receptor-mediated endocytosis for use in transmembrane transport of exogenous molecules. The folate receptor has a high affinity for folate, which, upon binding the folate receptor, impacts the cell cycle in dividing cells. As a result, folate receptors have been implicated in a variety of cancers which have been shown to demonstrate high folate receptor expression. For example, epithelial cancers of the ovary, mammary gland, colon, lung, nose, throat, and brain have all been reported to express elevated levels of the FR. In fact, greater than 90% of all human ovarian tumors are known to express large amounts of this receptor.

In contrast, folate receptor expression in normal tissues is limited (e.g., kidney, liver, intestines and placenta). This differential expression of the folate receptor in neoplastic and normal tissues has made the folate receptor an ideal target for the development of therapeutics and diagnostics. The development of folate conjugates represents one avenue for the discovery of therapeutics and diagnostics that has taken advantage of differential expression of the folate receptor with success. For example, radionuclide-chelators conjugated to folate have been used as non-invasive probes for diagnostic imaging purposes. One such imaging agent is of the formula III

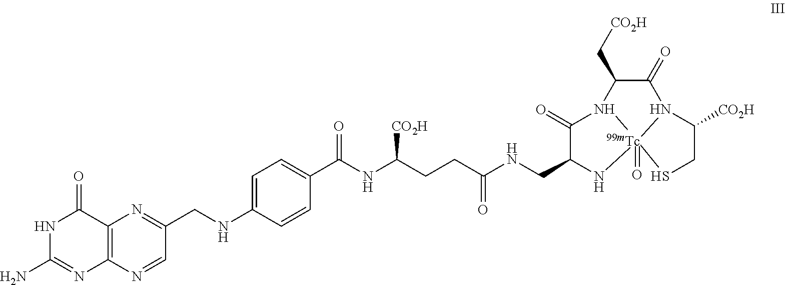

(also referred to herein as ^{99m}Tc-Compound IV) as described in WO03/092742, which is incorporated herein by reference. Compound (III) has found use as a cancer imaging agent as described in, for example, WO2011/014821. One of skill in the art will recognize that compound (III) can exist as syn- and anti-isomers in reference to the relative position of the Tc═O double bond.

Because imaging agent (III) is of interest in the area of cancer imaging, more efficient procedures for producing imaging agents having higher radioactive purity are desired.

Throughout this disclosure, various publications, patents and patent applications are referenced. The disclosures of these publications, patents and applications in their entireties are hereby incorporated by reference into this disclosure.

### SUMMARY

In some embodiments, the present disclosure provides an imaging agent composition comprising a targeting molecule, a chelating agent and a reducing agent. In some aspects of these embodiments, the targeting molecule is of the formula

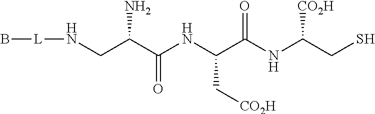

wherein B is a binding ligand and L is an optional linker; or a pharmaceutically acceptable salt thereof. In some aspects of these embodiments, B is a folate or a PSMA binding ligand. In some aspects of these embodiments, the optional linker L comprises at least one amino acid residue. In some aspects of these embodiments, the optional linker L comprises at least two amino acid residues.

In some aspects of these embodiments, the at least one chelating agent is selected from the group consisting of ethylene diamine tetraacetic acid (EDTA), disodium ethylene diamine tetraacetic acid dihydrate, gluconic acid, lactic acid, citric acid, sodium gluconate, sodium lactate, sodium citrate, potassium gluconate, potassium lactate and potassium citrate.

In some aspects of these embodiments, the chelating agent is a combination of sodium gluconate and disodium ethylene diamine tetraacetic acid dihydrate. In some aspects of these embodiments, the chelating agent is a combination of sodium gluconate and disodium ethylene diamine tetraacetic acid dihydrate in a ratio of about 25:1 to about 100:1 by weight or 25:1 to 100:1 by weight. In some aspects of these embodiments, the reducing agent is stannous chloride. In some aspects of these embodiments, the imaging agent composition has a pH in the range of about 6.5 to about 7.5 (or 6.5-7.5). In some aspects of these embodiments, the imaging agent composition has a pH in the range of about 6.5 to about 7.0 (or 6.5-7.0). In some aspects of these embodiments, the imaging agent composition has a pH of about 6.8 (or 6.8).

In other aspects of these embodiments, the imaging agent composition further comprises a radiolabel source. In some aspects of these embodiments, the radiolabel source is ^{99m}Tc-pertechnetate. In some aspects of these embodiments, the ^{99m}Tc-pertechnetate is in an amount in the range of about 1 to about 100 mCi/mg (or 1 to 100 mCi/mg). In some aspects of these embodiments, the ^{99m}Tc-pertechnetate is in an amount in the range of about 1 to about 50 mCi/mg (or 1 to 50 mCi/mg). In some aspects of these embodiments, the composition comprises a targeting molecule bound to a radiolabel source to provide an imaging agent of the formula

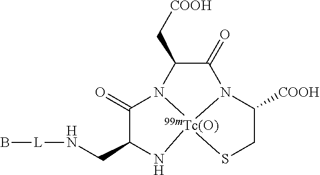

wherein B is a binding ligand and L is an optional linker; or a pharmaceutically acceptable salt thereof. In some aspects of these embodiments, B is a folate or a PSMA binding ligand. In some aspects of these embodiments, the optional linker L comprises at least one amino acid residue. In some aspects of these embodiments, the optional linker L comprises at least two amino acid residues.

In some embodiments, the present disclosure provides an imaging agent composition comprising a targeting molecule, a chelating agent and a reducing agent, wherein the targeting molecule comprises a compound of the formula II

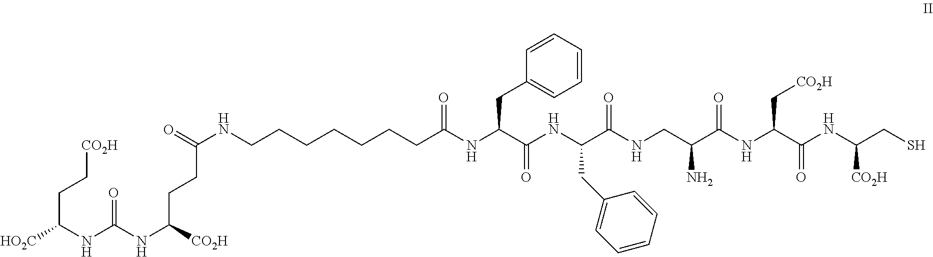

or a pharmaceutically acceptable salt thereof, or a compound of the formula IV

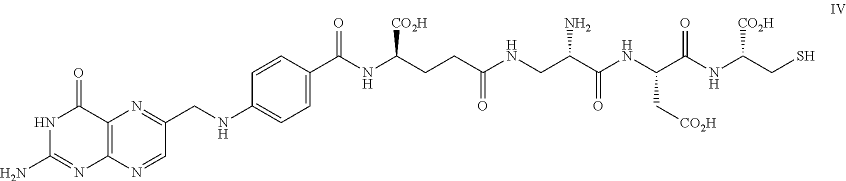 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof.

In some aspects of these embodiments, the at least one chelating agent is selected from the group consisting of ethylene diamine tetraacetic acid (EDTA), disodium ethylene diamine tetraacetic acid dihydrate, &conic acid, lactic acid, citric acid, sodium gluconate, sodium lactate, sodium citrate, potassium gluconate, potassium lactate and potassium citrate. In some aspects of these embodiments, the chelating agent is a combination of sodium gluconate and disodium ethylene diamine tetraacetic acid dihydrate. In some aspects of these embodiments, the chelating agent is a combination of sodium gluconate and disodium ethylene diamine tetraacetic acid dihydrate in a ratio of about 25:1 to about 100:1 by weight or 25:1 to 100:1 by weight. In some aspects of these embodiments, the reducing agent is stannous chloride. In some aspects of these embodiments, the imaging agent composition has a pH in the range of about 6.5 to about 7.5 (or 6.5-7.5). In some aspects of these embodiments, the imaging agent composition has a pH in the range of about 6.5 to about 7.0 (or 6.5-7.0). In some aspects of these embodiments, the imaging agent composition has a pH of about 6.8 (or 6.8).

In other aspects of these embodiments, the imaging agent composition further comprises a radiolabel source. In some aspects of these embodiments, the radiolabel source is ^{99m}Tc-pertechnetate. In some aspects of these embodiments, the ^{99m}Tc-pertechnetate is in an amount in the range of about 1 to about 100 mCi/mg (or 1 to 100 mCi/mg). In some aspects of these embodiments, the ^{99m}Tc-pertechnetate is in an amount in the range of about 1 to about 50 mCi/mg (or 1 to 50 mCi/mg).

In some embodiments, the present disclosure provides an imaging agent composition comprising a targeting molecule, a chelating agent and a reducing agent, wherein the targeting molecule comprises a compound of the formula II

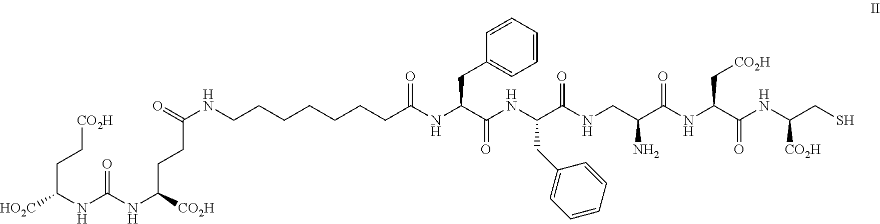

or a pharmaceutically acceptable salt thereof. In some aspects of these embodiments, the at least one chelating agent is selected from the group consisting of ethylene diamine tetraacetic acid (EDTA), disodium ethylene diamine tetraacetic acid dihydrate, gluconic acid, lactic acid, citric acid, sodium gluconate, sodium lactate, sodium citrate, potassium gluconate, potassium lactate and potassium citrate. In some aspects of these embodiments, the chelating agent is a combination of sodium gluconate and disodium ethylene diamine tetraacetic acid dihydrate. In some aspects of these embodiments, the chelating agent is a combination of sodium gluconate and disodium ethylene diamine tetraacetic acid dihvdrate in a ratio of about 25:1 to about 100:1 by weight or 25:1 to 100:1 by weight. In some aspects of these embodiments, the reducing agent is stannous chloride. In some aspects of these embodiments, the imaging agent composition has a pH in the range of about 6.5 to about 7.5 (or 6.5-7.5). In some aspects of these embodiments, the imaging agent composition has a pH in the range of about 6.5 to about 7.0 (or 6.5-7.0). In some aspects of these embodiments, the imaging agent composition has a pH of about 6.8 (or 6.8).

In other aspects of these embodiments, the imaging agent composition further comprises a radiolabel source. In some aspects of these embodiments, the radiolabel source is ^{99m}Tc-pertechnetate. In some aspects of these embodiments, the ^{99m}Tc-pertechnetate is in an amount in the range of about 1 to about 100 mCi/mg (or 1 to 100 mCi/mg). In some aspects of these embodiments, the ^{99m}Tc-pertechnetate is in an amount in the range of about 1 to about 50 mCi/mg (or 1 to 50 mCi/mg).

In some embodiments, the present disclosure provides an imaging agent composition comprising a targeting molecule, a chelating agent and a reducing agent, wherein the targeting molecule comprises a compound of the formula IV

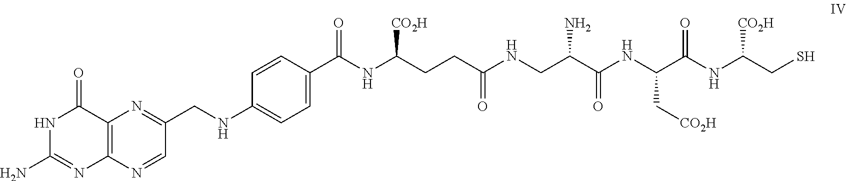 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof. In some aspects of these embodiments, the at least one chelating agent is selected from the group consisting of ethylene diamine tetraacetic acid (EDTA), disodium ethylene diamine tetraacetic acid dihydrate, gluconic acid, lactic acid, citric acid, sodium gluconate, sodium lactate, sodium citrate, potassium gluconate, potassium lactate and potassium citrate. In some aspects of these embodiments, the chelating agent is a combination of sodium gluconate and disodium ethylene diamine tetraacetic acid dihydrate. In some aspects of these embodiments, the chelating agent is a combination of sodium gluconate and disodium ethylene diamine tetraacetic acid dihydrate in a ratio of about 25:1 to about 100:1 by weight or 25:1 to 100:1 by weight. In some aspects of these embodiments, the reducing agent is stannous chloride. In some aspects of these embodiments, the imaging agent composition has a pH in the range of about 6.5 to about 7.5 (or 6.5-7.5). In some aspects of these embodiments, the imaging agent composition has a pH in the range of about 6.5 to about 7.0 (or 6.5-7.0). In some aspects of these embodiments, the imaging agent composition has a pH of about 6.8 (or 6.8).

In other aspects of these embodiments, the imaging agent composition further comprises a radiolabel source. In some aspects of these embodiments, the radiolabel source is ^{99m}Tc-pertechnetate. In some aspects of these embodiments, the ^{99m}Tc-pertechnetate is in an amount in the range of about 1 to about 100 mCi/mg (or 1 to 100 mCi/mg). In some aspects of these embodiments, the ^{99m}Tc-pertechnetate is in an amount in the range of about 1 to about 50 mCi/mg (or 1 to 50 mCi/mg).

In other embodiments, the disclosure provides a lyophilized imaging agent composition comprising two or more chelating agents selected from the group consisting of ethylene diamine tetraacetic acid, disodium ethylene diamine tetraacetic acid dihydrate, gluconic acid, lactic acid, citric acid, sodium gluconate, sodium lactate, sodium citrate, potassium gluconate, potassium lactate and potassium citrate, and a reducing agent, wherein the targeting molecule comprises a compound of the formula II

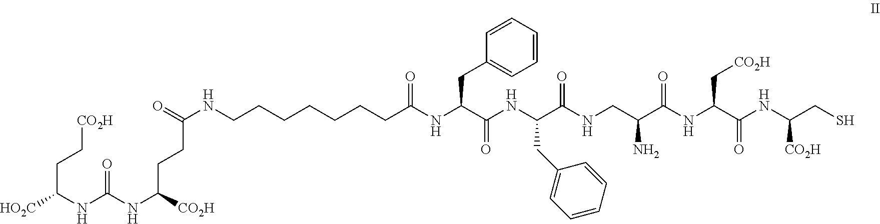

or a pharmaceutically acceptable salt thereof, and the reducing agent is stannous chloride.

In some aspects of these embodiments, the two or more chelating agents are disodium ethylene diamine tetraacetic acid dihydrate and sodium gluconate. In some aspects of these embodiments, the disodium ethylene diamine tetraacetic acid dihydrate and the sodium gluconate are in a ratio of about 25:1 and to about 100:1 by weight (or 25:1 to 100:1 by weight).

In other embodiments, the disclosure provides a kit comprising a first vial comprising a lyophilized imaging agent composition comprising a targeting molecule, two or more chelating agents selected from the group consisting of ethylene diamine tetraacetic acid, disodium ethylene diamine tetraacetic acid dihydrate, gluconic acid, lactic acid, citric acid, sodium gluconate, sodium lactate, sodium citrate, potassium gluconate, potassium lactate, and potassium citrate, and a reducing agent, wherein the targeting molecule comprises a compound of the formula II

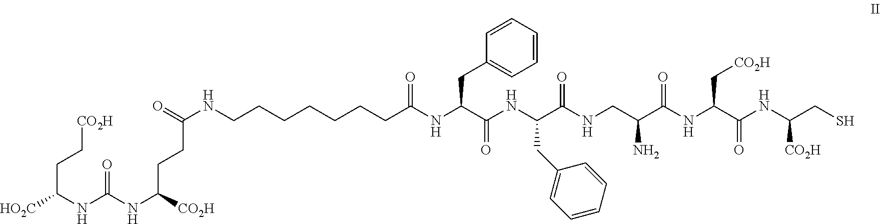

or a pharmaceutically acceptable salt thereof, and the reducing agent is stannous chloride.

In some aspects of these embodiments, the kit further comprises a second vial comprising an aqueous solution of ^{99m}Tc-pertechnetate.

In other embodiments, the disclosure provides a method for preparing an imaging agent composition comprising the steps of

(a) preparing a first solution comprising aqueous stannous chloride;

(b) preparing a second solution comprising aqueous stannous chloride, sodium gluconate and disodium ethylene diamine, tetraacetic acid dihydrate by contacting the first solution with sodium gluconate and disodium ethylene diamine tetraacetic acid dihydrate in a vessel to form the second solution;

(c) preparing a third solution comprising aqueous stannous chloride, sodium gluconate disodium ethylene diamine tetraacetic acid dihydrate, and a compound of the formula II

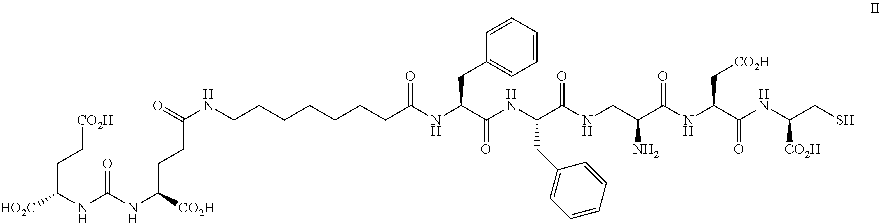

or a pharmaceutically acceptable salt thereof, by contacting the second solution with the compound of the formula

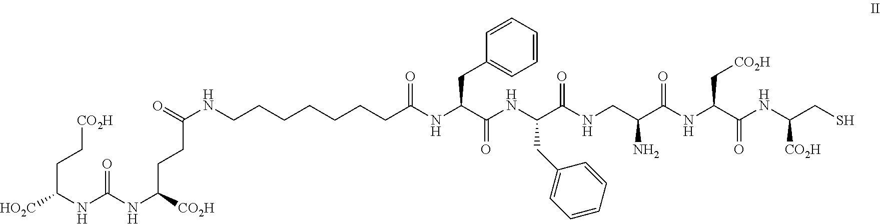

or a pharmaceutically acceptable salt thereof;

(d) adjusting the pH of the third solution to a pH in the range of about 6.5 to about 7.5 (or 6.5-7.5); and

(e) lyophilizing the third solution to form a lyophilized imaging agent composition.

In some aspects of these embodiments, the method further comprises the step of contacting the lyophilized imaging agent composition with an aqueous solution of ^{99m}Tc-pertechnetate.

In some aspects of these embodiments, the step of contacting the lyophilized imaging agent composition with an aqueous solution of ^{99m}Tc-pertechnetate is conducted at a temperature of about 17° C. to about 27° C. (or 17° C-27° C.).

Embodiments of the disclosure are further described by the following enumerated clauses:

1. An imaging agent composition comprising a targeting molecule, a chelating agent and a reducing agent, wherein the targeting molecule is of the formula

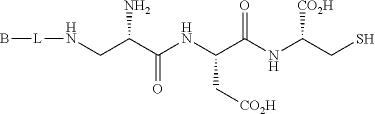

or a pharmaceutically acceptable salt thereof, wherein B is a binding ligand and L is an optional linker.

2. The imaging agent composition of clause 1, wherein B is a folate or a PSMA binding ligand.

3. The imaging agent composition of clause 1 or 2, wherein the optional linker L comprises at least one amino acid residue.

4. The imaging agent composition of any one of clauses 1 to 3, wherein the optional linker L comprises at least two amino acid residues.

5. The imaging agent composition of any one of clauses 1 to 4, wherein the at least one chelating agent is selected from the group consisting of ethylene diamine tetraacetic acid, disodium ethylene diamine tetraacetic acid dihydrate, glitconic acid, lactic acid, citric acid, sodium gluconate, sodium lactate, sodium citrate, potassium gluconate, potassium lactate and potassium citrate.

6. The imaging agent composition of any one of clauses 1 to 5, wherein the wherein the chelating agent is a combination of sodium gluconate and disodium ethylene diamine tetraacetic acid dihydrate.

7. The imaging agent composition of any one of clauses 1 to 6, wherein the chelating agent is a combination of sodium gluconate and di sodium ethylene diamine tetraacetic acid dihydrate in a ratio of about 25:1 to about 100:1 by weight.

8. The imaging agent composition of any one of clauses 1 to 7, wherein the reducing agent is stannous chloride.

9. The imaging agent composition of any one of clauses 1 to 8, having a pH in the range of about 6.5 to about 7.5.

10. The imaging agent composition of any one of clauses 1 to 9, having a pH in the range of about 6.5 to about 7.0.

11. The imaging agent composition of any one of clauses 1 to 10, having a pH of about 6.8.

12. The imaging agent composition of any one of clauses 1 to 11, further comprising a radiolabel source.

13. The imaging agent composition of clause 12, wherein the radiolabel source is ^{99m}Tc-pertechneate.

14. The imaging agent composition of clause 13, wherein the targeting molecule and the radiolabel source combine to form an imaging agent of the formula

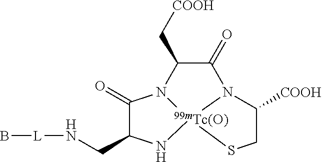

or a pharmaceutically acceptable salt thereof, wherein B is a binding ligand and L is an optional linker.

15. The imaging agent composition of clause 14, wherein the ^{99m}Tc-pertechnetate is in an amount in the range of about 1 mCi/mg to about 100 mCi/mg.

16. The imaging agent composition of clause 15, wherein the ^{99m}Tc-pertechnetate is in an amount in the range of about 1 mCi/mg to about 50 mCi/mg.

17. The imaging agent composition of any one of clauses 1 to 16, wherein the targeting molecule comprises a compound of the formula

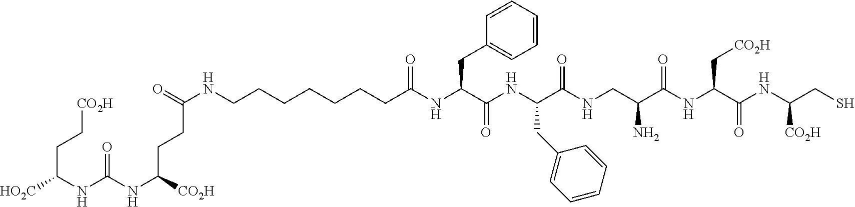

or a pharmaceutically acceptable salt thereof. 18. The imaging agent composition of any one of clauses 1 to 16, wherein the targeting molecule comprises a compound of the formula

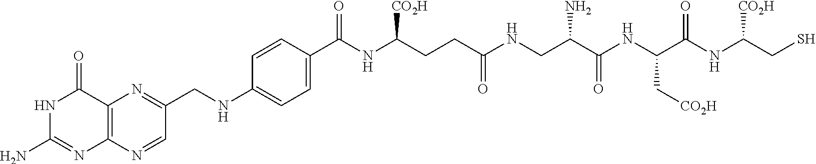 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof.

19. An imaging agent composition comprising a targeting molecule, or a pharmaceutically acceptable salt thereof, a chelating agent and a reducing agent, wherein the chelating agent is a combination of sodium gluconate and disodium ethylene diamine tetraacetic acid dihydrate, the reducing agent is stannous chloride, and the imaging agent composition has a pH in the range of about 6.5 to about 7.5.

20. The imaging agent composition of clause 19, wherein the chelating agent is a combination of sodium gluconate and di sodium ethylene diamine tetraacetic acid dihydrate in a ratio of about 25:1 to about 100:1 by weight.

21. The imaging agent composition of clause 19 to 20, having a pH of about 6.8.

22. The imaging agent composition of any one of clauses 19 to 21, further comprising a radiolabel source.

23. The imaging agent composition of clause 22, wherein the radiolabel source is ^{99m}Tc-pertechneate.

24. A lyophilized imaging agent composition comprising a targeting molecule, two or more chelating agents selected from the group consisting of ethylene diamine tetraacetic acid, disodium ethylene diamine tetraacetic acid dihydrate, glitconic acid, lactic acid, citric acid, sodium gluconate, sodium lactate, sodium citrate, potassium gluconate, potassium lactate and potassium citrate, and a reducing agent, wherein the targeting molecule comprises a compound of the formula

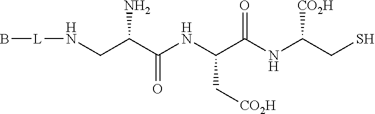

or a pharmaceutically acceptable salt thereof, wherein B is a binding ligand and L is an optional linker, and wherein the reducing agent is stannous chloride.

25. The lyophilized imaging agent composition of clause 24, wherein B is a folate or a PSMA binding ligand.

26. The lyophilized imaging agent composition of clause 24 or 25, wherein the optional linker L comprises at least one amino acid residue.

27. The lyophilized imaging agent composition of any one of claims 24 to 26, wherein the optional linker L comprises at least two amino acid residues.

28. The lyophilized imaging agent composition of clause 27, wherein the two or more chelating agents are disodium ethylene diamine tetraacetic acid dihydrate and sodium gluconate.

29. The lyophilized imaging agent composition of clause 28, wherein the disodium ethylene diamine tetraacetic acid dihydrate and the sodium gluconate are in a ratio of about 25:1 to about 100:1 by weight.

30. A lyophilized imaging agent composition comprising a targeting molecule, two or more chelating agents selected from the group consisting of ethylene diamine tetraacetic acid, disodium ethylene diamine tetraacetic acid dihydrate, giuconic acid, lactic acid, citric acid, sodium gluconate, sodium lactate, sodium citrate, potassium gluconate, potassium lactate and potassium citrate. and a reducing agent, wherein the targeting molecule comprises a compound of the formula

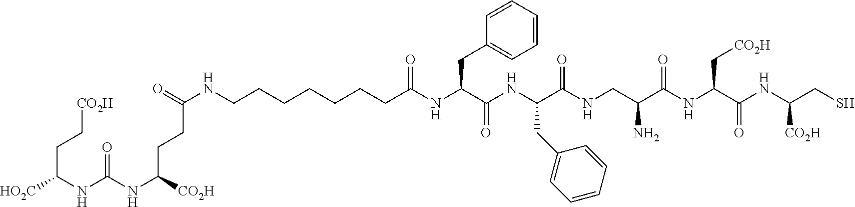

or a pharmaceutically acceptable salt thereof, and wherein the reducing agent is stannous chloride.

31. The lyophilized imaging agent composition of clause 30, wherein the two or more chelating agents are disodium ethylene diamine tetraacetic acid dihydrate and sodium gluconate.

32. The lyophilized imaging agent composition of clause 31, wherein the disodium ethylene diamine tetraacetic acid dihydrate and the sodium gluconate are in a ratio of about 25:1 to about 100:1 by weight.

33. A lyophilized imaging agent composition comprising a targeting molecule, two or more chelating agents selected from the group consisting of ethylene diamine tetraacetic acid, disodium ethylene diamine tetraacetic acid dihydrate. &conic acid, lactic acid, citric acid, sodium gluconate, sodium lactate, sodium citrate, potassium gluconate, potassium lactate and potassium citrate, and a reducing agent, wherein the targeting molecule comprises a compound of the formula

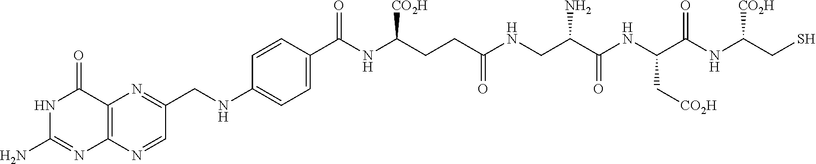 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, and wherein the reducing agent is stannous chloride.

34. The lyophilized imaging agent composition of clause 33, wherein the two or more chelating agents are disodium ethylene diamine tetraacetic acid dihydrate and sodium gluconate.

35. The lyophilized imaging agent composition of clause 34, wherein the disodium ethylene diamine tetraacetic acid dihydrate and the sodium gluconate are in a ratio of about 25:1 to about 100:1 by weight.

36. An imaging agent kit comprising a first vial comprising the lyophilized imaging agent of any one of clauses 24 to 35.

37. The kit of clause 36, further comprising a second vial comprising an aqueous solution of ^{99m}Tc-pertechnetate.

38. A method for preparing an imaging agent composition comprising the steps of

(a) preparing a first solution comprising aqueous stannous chloride;

(b) preparing a second solution comprising aqueous stannous chloride, sodium gluconate and disodium ethylene diamine tetraacetic acid dihydrate by contacting the first solution with sodium gluconate and disodiurn ethylene diamine tetraacetic acid dihydrate in a vessel to form the second solution;

(c) preparing a third solution comprising aqueous stannous chloride, sodium gluconate disodium ethylene diamine tetraacetic acid dihydrate, and a compound of the formula

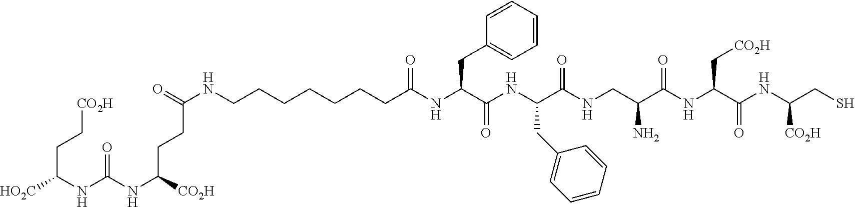

or a pharmaceutically acceptable salt thereof, by contacting the second solution with the compound of the formula

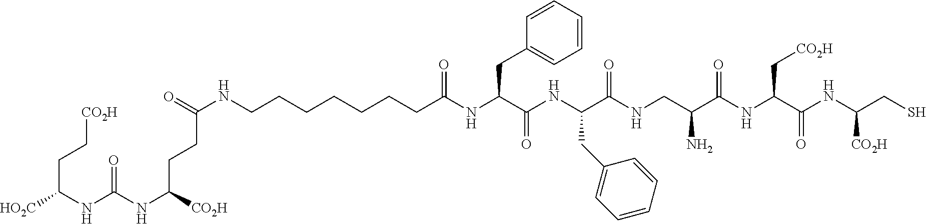

or a pharmaceutically acceptable salt thereof;

(d) adjusting the pH of the third solution to a pH in the range of about 6.5 to about 7.5; and

(e) lyophilizing the third solution to form a lyophilized imaging agent composition.

39. The method of clause 38, further comprising the step of contacting the lyophilized imaging agent composition with an aqueous solution of ^{99m}Tc-pertechnetate.

40. The method of clause 39, wherein the step of contacting the lyophilized imaging agent composition with an aqueous solution of ^{99m}Tc-pertechnetate is conducted at a temperature of about 17° C. to about 27° C.

41. A method for preparing an imaging agent composition comprising the steps of

(a) preparing a first solution comprising aqueous stannous chloride;

(b) preparing a second solution comprising aqueous stannous chloride, sodium gluconate and disodium ethylene diamine tetraacetic acid dihydrate by contacting the first solution with sodium gluconate and disodium ethylene diamine tetraacetic acid dihydrate in a vessel to form the second solutiom

(c) preparing a third solution comprising aqueous stannous chloride, sodium gluconate disodium ethylene diamine tetraacetic acid dihydrate, and a compound of the formula

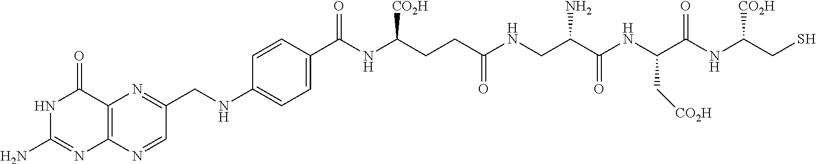 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof, by contacting the second solution with the compound of the formula

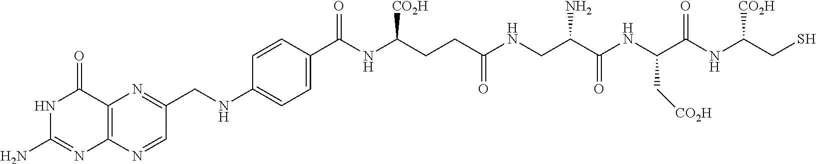 [SMILES: Nc1nc2ncc(CNc3ccc(C(=O)N[C@H](CCC(=O)NC[C@H](N)C(=O)N[C@@H](CC(=O)O)C(=O)N[C@@H](CS)C(=O)O)C(=O)O)cc3)nc2c(=O)[nH]1]

or a pharmaceutically acceptable salt thereof;

(d) adjusting the pH of the third solution to a pH in the range of about 6.5 to about 7.5; and

(e) lyophilizing the third solution to form a lyophilized imaging agent composition.

42. The method of clause 41, further comprising the step of contacting the lyophilized imaging agent composition with an aqueous solution of ^{99m}Tc-pertechnetate.

43. The method of clause 42, wherein the step of contacting the lyophilized imaging agent composition with an aqueous solution of ^{99m}Tc-pertechnetate is conducted at a temperature of about 17° C. to about 27° C.

### BRIEF DESCRIPTION OF THE DRAWINGS

FIG. 1 shows the radio-HPLC profile of ^{99m}Tc-Compound II prepared by reconstituting inventive formulation kit at room temperature taken immediately after labelling and showing a radiochemical purity of 95.5%.

FIG. 2 shows the TLC determination of radiochemical purity: 2A shows Instant Thin Layer Chromatography-Silica Gel (ITLC-SG) plate developed by saturated sodium chloride solution to detect free ^{99m}Tc-pertechnetate and ^{99m}Tc-gluconate/EDTA. 2B shows ITLC-SG plate developed by 0.1% sodium dibasic phosphate solution to detect reduced-hydrolyzed colloidal ^{99m}Tc.

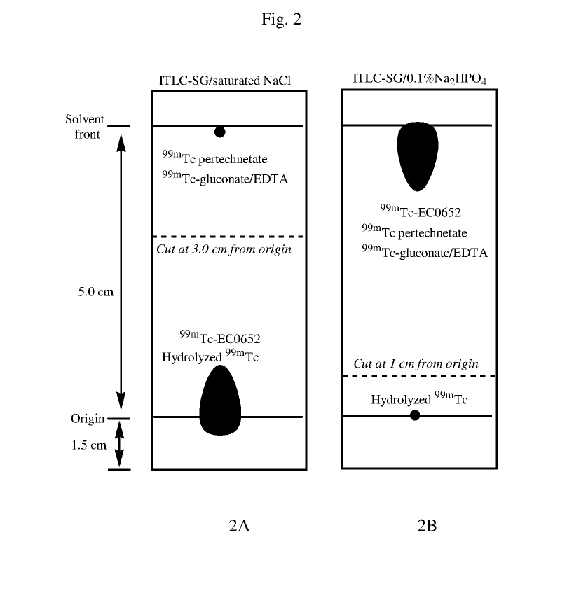

FIG. 3 shows the radio-HPLC profile of ^{99m}Tc-Compound II prepared by reconstituting an Example DC 1A kit vial (comparative example) and incubating at room temperature provided a radiochemical purity of 84%.

FIG. 4 shows the radio-HPLC profile of ^{99m}Tc-Compound II prepared by reconstituting an Example DC1B kit vial (inventive example) and incubating at room temperature provided a radiochemical purity of 98%.

FIG. 5 shows the radio-HPLC profile of ^{99m}Tc-Compound IV prepared by reconstituting an Example DC2A kit vial (comparative example) and incubating at room temperature provided a radiochemical purity of 82.5%.

FIG. 6 shows the radio-HPLC profile of ^{99m}Tc-Compound IV prepared by reconstituting an Example DC2B kit vial (inventive example) and incubating at room temperature provided a radiochemical purity of 94.2%.

### DEFINITIONS

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like.

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like.

As used herein, the term “heteroaryl” refers to a monocyclic or fused ring group of 5 to 12 ring atoms containing one, two, three or four ring heteroatoms selected from the group consisting of nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like.

As used herein, “hydroxy” or “hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “trihalomethyl” refers to a methyl group having three halo substituents, such as a trifluoromethyl group.

As used herein, “cyano” refers to a —CN group.

As used herein, “sulfinyl” refers to a —S(O)R″ group, where R″ is any R group as described in the various embodiments provided herein, or R″ may be a hydroxyl group.

As used herein, “sulfonyl” refers to a —S(O)₂R″ group, where R″ is any R group as described in the various embodiments provided herein, or R″ may be a hydroxyl group.

As used herein, “S-sulfonamido” refers to a —S(O)₂NR″R″ group, where R″ is any R group as described in the various embodiments provided herein.

As used herein, “N-sulfonamido” refers to a —NR″S(O)₂R″ group, where R″ is any R group as described in the various embodiments provided herein.

As used herein, “O-carbamyl” refers to a —OC(O)NR″R″ group, where R″ is any R group as described in the various embodiments provided herein.

As used herein, “N-carbamyl” refers to an R″OC(O)NR″-group, where R″ is any R group as described in the various embodiments provided herein.

As used herein, “O-thiocarbamyl” refers to a —OC(S)NR″R″ group, where R″ is any R group as described in the various embodiments provided herein.

As used herein, “N-thiocarbamyl” refers to a R″OC(S)NR″-group, where R″ is any R group as described in the various embodiments provided herein.

As used herein, “amino” refers to an —NR″R″ group, where R″ is any R group as described in the various embodiments provided herein.

As used herein, “C-amido” refers to a —C(O)NR″R″ group, where R″ is any R group as described in the various embodiments provided herein.

As used herein, “N-amido” refers to a R″C(O)NR″-group, where R″ is any R group as described in the various embodiments provided herein.

As used herein, “nitro” refers to a —NO₂ group.

As used herein, “bond” refers to a covalent bond.

As used herein, “optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the term “pharmaceutically acceptable salt” refers to those salts which counter ions which may be used in pharmaceuticals. Such salts include:

(1) acid addition salts, which can be obtained by reaction of the free base of the parent conjugate with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or

(2) salts formed when an acidic proton present in the parent conjugate either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein.

As used herein, “amino acid” (a.k.a. “AA”) means any molecule that includes an alpha-carbon atom covalently bonded to an amino group and an acid group. The acid group may include a carboxyl group. “Amino acid” may include molecules having one of the formulas:

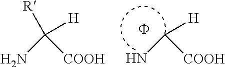

wherein R′ is a side group and Φ includes at least 3 carbon atoms. “Amino acid” includes stereoisomers such as the D-amino acid and L-amino acid forms. Illustrative amino acid groups include, but are not limited to, the twenty endogenous human amino acids and their derivatives, such as lysine (Lys), asparagine (Asn), threonine (Thr), serine (Ser), isoleucine (Ile), methionine (Met), proline (Pro), histidine (His), glutamine (Gln), arginine (Arg), glycine (Gly), aspartic acid (Asp), glutamic acid (Glu), alanine (Ala), valine (Val), phenylalanine (Phe), leucine (Leu), tyrosine (Tyr), cysteine (Cys), tryptophan (Trp), phosphoserine (PSER), sulfo-cysteine, arginosuccinic acid (ASA), hydroxyproline, phosphoethanolamine (PEA), sarcosine (SARC), taurine (TAU), carnosine (CARN), citrulline (CIT), anserine (ANS), 1,3-methyl-histidine (ME-HIS), alpha-amino-adipic acid (AAA), beta- alanine (BALA), ethanolamine (ETN), gamma-amino-butyric acid (GABA), beta-amino- isobutyric acid (BAIA), alpha-amino-butyric acid (BABA), L-allo-cystathionine (cystathionine- A; CYSTA-A), L-cystathionine (cystathionine-B; CYSTA-B), cystine, allo-isoleucine (ALLO- ILE), DL-hydroxylysine (hydroxylysine (I)), DL-allo-hydroxylysine (hydroxylysine (2)), ornithine (ORN), homocystine (HCY), and derivatives thereof. It will be appreciated that each of these examples are also contemplated in connection with the present disclosure in the D-configuration as noted above. Specifically, for example, D-lysine (D-Lys), D-asparagine (D-Asn), D-threonine (D-Thr), D-serine (D-Ser), D-isoleucine (D-Ile), D-methionine (D-Met), D-proline (D-Pro), D-histidine (D-His), D-glutamine (D-Gln), D-arginine (D-Arg), D-glycine (D-Gly), D-aspartic acid (D-Asp), D-glutamic acid (D-Glu), D-alanine (D-Ala), D-valine (D-Val), D-phenylalanine (D-Phe), D-leucine (D-Leu), D-tyrosine (D-Tyr), D-cysteine (D-Cys), D-tryptophan (D-Trp), D-citrulline (D-CIT), D-carnosine (D-CARN), and the like. In connection with the embodiments described herein, amino acids can be covalently attached to other portions of the conjugates described herein through their alpha-amino and carboxy functional groups (i.e. in a peptide bond configuration), or through their side chain functional groups (such as the side chain carboxy group in glutamic acid) and either their alpha-amino or carboxy functional groups. It will be understood that amino acids, when used in connection with the conjugates described herein, may exist as zwitterions in a conjugate in which they are incorporated.

### DETAILED DESCRIPTION

The present disclosure provides improved formulations of imaging agents. In one embodiment, the present disclosure provides formulations, as described herein, of a compound of the formula I formula III for radio-imaging applications in a subject. Further, in another embodiment, the present disclosure provides formulations, as described herein, of a compound of the formula II or formula IV for radiolabelling with ^{99m}Tc. In some embodiments, liquid formulations of a compound of the formula II or formula IV described herein are lyophilized, or freeze-dried, by first exposing opened vials of the formulations to lyophilization to effect sublimation of water from the samples. The resulting products can be a powder or cake which upon sealing with a stopper and seal can be stored for extended periods and shipped to the end user while maintaining activity and stability. A formulation cake can be reconstituted, for example, just prior to time of use by rehydration of the cake with an aqueous solution such as water for injection, buffer or other diluent suitable for pharmaceutical use. Following reconstitution and gentle admixture, and labeling with ^{99m}Tc, the solution is ready to be administered to the subject.

In particular, the formulations described herein contemplate use of excipients, for example chelating agents and reducing agents, in admixture with a targeting molecule (e.g. a compound of the formula II) at a selected range of pH, which composition can be lyophilized. It will be appreciated that stability of the lyophilized formulation is greater than that of the corresponding liquid formulation. It has been discovered that the formulations described herein provide for more efficient low-temperature radiolabelling of a targeting molecule, (e.g. of the formula II or formula IV) with, for example, ^{99m}Tc to provide a labelled compound of the formula I with high radiopurity.

Typical methods known in the art for labelling with ^{99m}Tc include, but are not limited to, the reduction of pertechnetate ions in the presence of a chelating precursor to form the labile ^{99m}Tc-precursor complex, which, in turn, reacts with a metal binding group of a bifunctionally modified conjugate (e.g. Compound II or Compound IV) to form a ^{99m}Tc-conjugate (e.g. ^{99m}Tc-Compound II or ^{99m}Tc-Compound IV). The reducing agent can be, for example, SnCl₂. Stannous ion is readily available as its dehydrate (such as tin chloride dihydrate, SnCl₂.2H₂O), or it can be generated in situ from tin metal (such as foil, granules, powder, turnings and the like) by contacting with aqueous acid (such as HCl). The stannous ion solution can be prepared by dissolving SnCl₂.2H₂O in aqueous HCl at a concentration preferred for a particular application.

In some embodiments, optional stabilizing agents and excipients can be added to the formulations described herein. Examples of excipients include, but are not limited to, vinyl polymers, polyoxyethylene-polyoxypropylene polymers or co-polymers, sugars or sugar alcohols, polysaccharides, proteins, poly(ethyleneoxide), and acrylamide polymers and derivatives or salts thereof, such as polyethylene glycol (or PEG), propylene glycol and polysorbate 80 (TWEEN). Vinyl polymers useful in connection with the disclosed formulations can be any conventional vinyl polymer known in the art as an excipient such as polyacrylic acid, polymethacrylic acid, polyvinyl pyrrolidone or polyvinyl alcohol. Sugars useful in connection with the disclosed formulations include tetroses, pentoses, hexoses, heptoses, octoses and nonoses, especially erythrose, threose, arabinose, lyxose, xylose, ribose, rhatnnose, fuxose, digitalose, quinovose, apiose, glucose, mannose, galaktose, fructose, sorbose, gulose, talose, allose, altrose, idose and glucoheptulose. Deoxy compounds like 3-deoxyglycose, amino compounds like glucosamine, ether compounds like 3-o-methylglucose and 3-o-butylglucose may also be used. Also contemplated as useful in connection with the disclosed formulations are sugar alcohols of any of the above, such as mannitol. Polysaccharides useful in connection with the disclosed formulations include cellulose or cellulose derivatives, glycosamino-glycans, agar, pectin, alginic acid, dextran, starch and chitosan. Glycosaminoglycans useful in connection with the disclosed formulations include hyaluronic acid, chondroitin, and the like. Cellulose derivatives include but are not limited to alkyl cellulose and hydroxy alkyl cellulose, for example, methyl cellulose, hydroxyethyl cellulose, carboxymethyl cellulose, hydroxypropyl-methyl cellulose and hydroxypropyl cellulose. Excipients can be employed at concentrations advantageous to the formulations described herein, such as in a range of about 0.04 mg to about 100 mg (or 0.04 mg to 100 mg) excipient per 4.0 mg targeting molecule.

It will be understood that stabilizing agents for the stannous ion may be present in the formulations described herein. For example, ascorbate (ascorbic acid) can improve specific loading of a chelator with reduced ^{99m}Tc-pertechnetate and minimize formation of Tc0₂, when the reducing agent is stannous ion. Other polycarboxylic acids, such as tartrate, citrate, phthalate, iminodiacetate, ethylenediaminetetraacetic acid (EDTA), diethylenetriaminepentaacetic acid (DTPA) and tricine, and the like, can also be used. Furthermore, it will be underrstood that any of a variety of anionic and/or hydroxylic oxygen-containing species can serve as stabilizing agents. For example in some embodiments, additional optional stabilizing agents can be salicylates, acetylacetonates, hydroxyacids, catechols, glycols and other polyols, such as glucoheptonate, and the like.

In some embodiments, B is a folate. In some embodiments, B is of the formula I

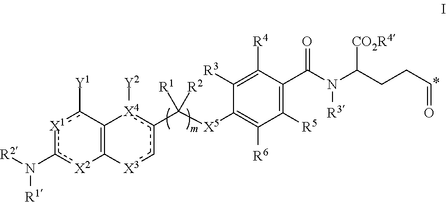

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R⁷, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R⁹, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R⁷, R⁸, R⁸, R⁹, R^{9′}, R¹⁰ and R^{10′} is independently H, D, C₁-C₆ alkyl, C₂-C₆ alkenyl or C₂-C₆ alkynyl;

X¹ is —NR¹¹—, ═N—, —N═, —C(R¹¹)═ or ═C(R¹¹)—;

X² is —NR^{11′}— or ═N—;

X³ is —NR^{11″}—, —N═ or —C(R^{11′})═;

X⁴ is —N═ or —C═;

X⁵ is NR¹² or CR¹²R^{12′};

Y¹ is H, D, —OR¹³, —SR¹³ or —NR¹³R^{13′} when X¹ is —N═ or —C(R¹¹)═, or Y¹ is ═O when X¹ is —NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is —N═;

R^{1′}, R^{2′}, R^{3′}, R^{4′}, R₁₁, R^{11′}, R_{11″}, R¹², R^{12′}, R¹³, R^{13′}, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl;

m is 1, 2, 3 or 4; and

* is a covalent bond.

In some embodiments, B is if the formula

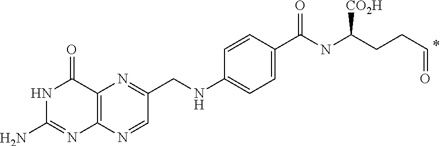 [SMILES: Nc1nc2ncc(CNc3ccc(C(=O)N[C@H](CCC=O)C(=O)O)cc3)nc2c(=O)[nH]1]

wherein * is a covalent bond.

In some embodiments, B is a PSMA binding ligand, such as those described in International Patent Publication WO2014/078484, incorporated herein by reference. In some embodiments, B comprises a urea or thiourea of D-lysine and one or more the following:

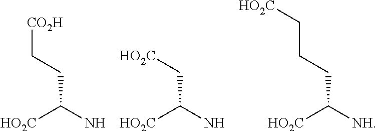

In some embodiments, B is a derivative of pentanedioic acid. Illustratively, the pentanedioic acid derivative is a compound of the formula:

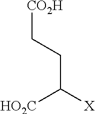

as described in U.S. Pat. No. 5,968,915, U.S. Pat. No. 5,863,536, U.S. Pat. No. 5,795,877, U.S. Pat. No. 5,962,521 and U.S. Pat. No. 5,902,817, each of which is incorporated herein by reference.

Illustrative PSMA ligands described in U.S. Pat. No. 5,968,915 include 2-[[methylhydroxyphosphinyl]methyl]pentanedioic acid; 2-[[ethylhydroxyphosphinyl]methyl]-pentanedioic acid; 2-[[propylhydroxyphosphinyl]methyl]pentanedioic acid; 2-[[butylhydroxyphosphinyl]methyl]pentanedioic acid; 2-[[cyclohexylhydroxyphosphinyl]-methyl]pentanedioic acid; 2-[[phenylhydroxyphosphinyl]methyl]pentanedioic acid; 2-[[2-(tetrahydrofuranyl)hydroxyphosphinyl]methyl] pentanedioic acid; 2-[[(2-tetrahydropyranyl)-hydroxyphosphinyl]methyl] pentanedioic acid; 2-[[((4-pyridyl)methyl)hydroxyphosphinyl]-methyl] pentanedioic acid; 2-[[((2-pyridyl)methyl)hydroxyphosphinyl]methyl] pentanedioic acid; 2-[[(phenylmethyl)hydroxyphosphinyl]methyl] pentanedioic acid; 2-[[((2-phenylethyl)-methyl)hydroxyphosphinyl]methyl] pentanedioic acid; 2-[[((3-phenylpropyl)methyl)-hydroxyphosphinyl]methyl] pentanedioic acid; 2-[[((3-phenylbutyl)methyl)-hydroxyphosphinyl]methyl] pentanedioic acid; 2-[[((2-phenylbutyl)methyl)-hydroxyphosphinyl]methyl] pentanedioic acid; 2-[[(4-phenylbutyl)hydroxyphosphinyl]-methyl]pentanedioic acid; and 2-[[(aminomethyl)hydroxyphosphinyl]methyl]pentanedioic acid.

Illustrative PSMA ligands described in U.S. Pat. No. 5,863,536 include N-[methylhydroxyphosphinyl]glutamic acid; N-[ethylhydroxyphosphinyl]glutamic acid; N-[propylhydroxyphosphinyl]glutamic acid; N-[butylhydroxyphosphinyl]glutamic acid; N-[phenylhydroxyphosphinyl]glutamic acid; N-[(phenylmethyl)hydroxyphosphinyl]glutamic acid; N-[((2-phenylethyl)methyl)hydroxyphosphinyl]glutamic acid; and N-methyl-N-[phenylhydroxyphosphinyl]glutamic acid.

Illustrative PSMA ligands described in U.S. Pat. No. 5,795,877 include 2-[[methylhydroxyphosphinyl]oxy]pentanedioic acid; 2-[[ethylhydroxyphosphinyl]oxy]-pentanedioic acid; 2-[[propylhydroxyphosphinyl]oxy]pentanedioic acid; 2-[[butylhydroxyphosphinyl]oxy]pentanedioic acid; 2-[[phenylhydroxyphosphinyl]-oxy]pentanedioic acid; 2-[[((4-pyridyl)methyl)hydroxyphosphinyl]oxy]pentanedioic acid; 2-[[((2-pyridyl)methyl)hydroxyphosphinyl]oxy]pentanedioic acid; 2-[[(phenylmethyl)-hydroxyphosphinyl]oxy]pentanedioic acid; and 2[[((2-phenylethyl)methyl)hydroxyphosphinyl]-oxy] pentanedioic acid.

Illustrative PSMA ligands described in U.S. Pat. No. 5,962,521 include 2-[[(N-hydroxy)carbamoyl]methyl]pentanedioic acid; 2-[[(N-hydroxy-N-methyl)carbamoyl]-methyl]pentanedioic acid; 2-[[(N-butyl-N-hydroxy) carbamoyl]methyl]pentanedioic acid; 2-[[(N-benzyl-N-hydroxy)c arbamoyl]methyl]pentanedioic acid; 2-[[(N-hydroxy-N-phenyl)-carbamoyl]methyl]pentanedioic acid; 2-[[(N-hydroxy-N-2-phenylethyl)carbamoyl]-methyl]pentanedioic acid; 2-[[(N-ethyl-N-hydroxy) carbamoyl]methyl]pentanedioic acid; 2-[[(N-hydroxy-N-propyl)carbamoyl]methyl]pentanedioic acid; 2-[[(N-hydroxy-N-3-phenylpropyl)carbamoyl]methyl]pentanedioic acid; 2-[[(N-hydroxy-N-4-pyridyl) carbamoyl]methyl]pentanedioic acid; 2-[[(N-hydroxy)carboxamido]methyl]pentanedioic acid; 2-[[N-hydroxy (methyl) carboxamido]methyl]pentanedioic acid; 2-[[N-hydroxy (benzyl) carboxamido]methyl]pentanedioic acid; 2-[[N-hydroxy(phenyl)carboxamido]methyl]-pentanedioic acid; 2-[[N-hydroxy(2-phenylethyl)carboxamido]methyl]pentanedioic acid; 2-[[N-hydroxy(ethyl)carboxamido]methyl]pentanedioic acid; 2-[[N-hydroxy(propyl) carboxamido]-methyl]pentanedioic acid; 2-[[N-hydroxy (3-phenylpropyl) carboxamido]methyl]pentanedioic acid; and 2-[[N-hydroxy(4-pyridyl)carboxamido]methyl]pentanedioic acid.

Illustrative PSMA ligands described in U.S. Pat. No. 5,902,817 include 2-[(sulfinyl)methyl]pentanedioic acid; 2-[(methylsulfinyl)methyl]pentanedioic acid; 2-[(ethylsulfinyl)methyl]pentanedioic acid; 2-[(propylsulfinyl)methyl]pentanedioic acid; 2-[(butylsulfinyl)methyl]pentanedioic acid; 2-[(phenylsulfinyl]methyl]pentanedioic acid; 2-[[(2-phenylethyl)sulfinyl]methyl]pentanedioic acid; 2-[[(3-phenylprop yl)sulfinyl]methyl]-pentanedioic acid; 2-[[(4-pyridyl)sulfinyl]methyl]pentanedioic acid; 2-[(benzylsulfinyl)-methyl]pentanedioic acid; 2-[(sulfonyl)methyl]pentanedioic acid; 2-[(methylsulfonyl)methyl]-pentanedioic acid; 2-[(ethylsulfonyl)methyl]pentanedioic acid; 2-[(propylsulfonyl)methyl]-pentanedioic acid; 2-[(butylsulfonyl)methyl]pentanedioic acid; 2-[(phenylsulfonyl]methyl]-pentanedioic acid; 2-[[(2-phenylethyl)sulfonyl]methyl]pentanedioic acid; 2-[[(3-phenylpropyl)sulfonyl]methyl]pentanedioic acid; 2-[[(4-pyridyl) sulfonyl]methyl]pentanedioic acid; 2-[(benzylsulfonyl)methyl]pentanedioic acid; 2-[(sulfoximinyl)methyl]pentanedioic acid; 2-[(methylsulfoximinyl)methyl]pentanedioic acid; 2-[(ethylsulfoximinyl)methyl]pentanedioic acid; 2-[(propylsulfoximinyl)methyl]pentanedioic acid; 2-[(butylsulfoximinyl)methyl]-pentanedioic acid; 2-[(phenylsulfoximinyl]methyl]pentanedioic acid; 2-[[(2-phenylethyl)-sulfoximinyl]methyl]pentanedioic acid; 2-[[(3-phenylpropyl) sulfoximinyl]methyl]pentanedioic acid; 2-[[(4-pyridyl)sulfoximinyl]methyl]pentanedioic acid; and 2-[(benzylsulfoximinyl)-methyl]pentanedioic acid.

Pentanedioic acid derivatives described herein have been reported to have high binding affinity at PS MA, including but not limited to the following phosphonic and phosphinic acid derivatives

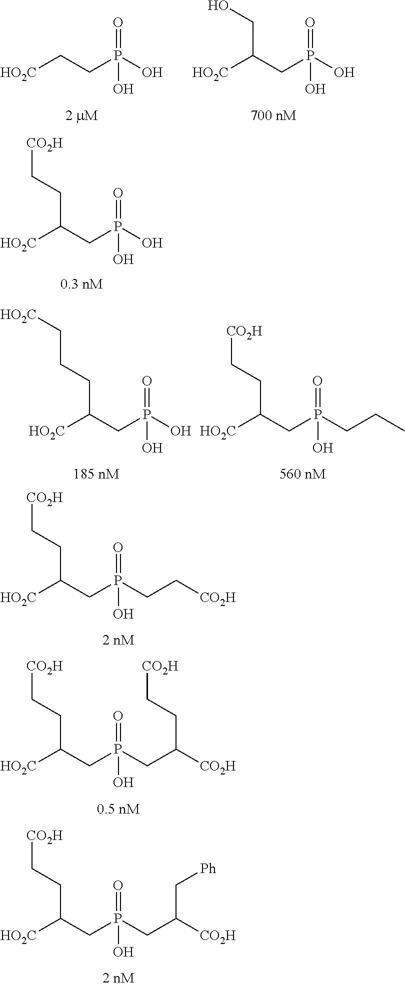

with the dissociation constants (Kᵢ values) shown for the E-I complex (see, Current Medicinal Chem. 8:949-0.957 (2001); Silverman, “The Organic Chemistry of Drug Design and Drug Action,” Elsevier Academic Press (2^{nd} Ed. 2003), the disclosures of which are incorporated herein by reference);

In another illustrative embodiment, the pentanedioic acid derivative includes a thiol group, such as compounds of the following formulae:

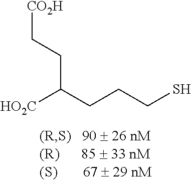 [SMILES: O=C(O)CCC(CCCS)C(=O)O]

with the inhibition constants (IC₅₀ values) shown for the E-I complex.

In another embodiment, the PSMA ligand is a urea of two amino acids. In one aspect, the amino acids include one or more additional carboxylic acids. In another aspect, the amino acids include one or more additional phosphoric, phosphonic, phosphinic, sulfinic, sulfonic, or boronic acids. In another aspect, the amino acids include one or more thiol groups or derivatives thereof. In another aspect, the amino acids include one or more carboxylic acid bioisosteres, such as tetrazoles and the like.

In some embodiments, the PSMA binding ligand includes at least four carboxylic acid groups, or at least three free carboxylic acid groups after the PSMA ligand is conjugated to the linker. It is understood that as described herein, carboxylic acid groups on the PSMA binding ligand include bioisosteres of carboxylic acids.

Illustratively, the PSMA binding ligand can be a compound of the formula

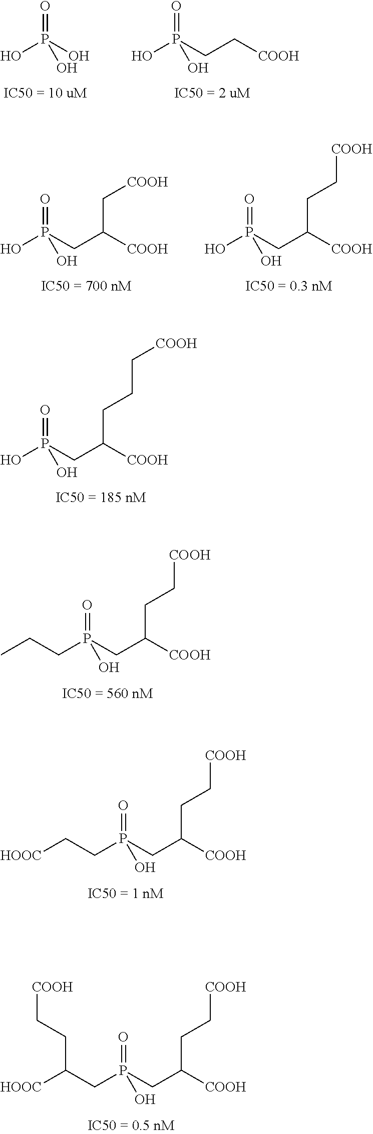

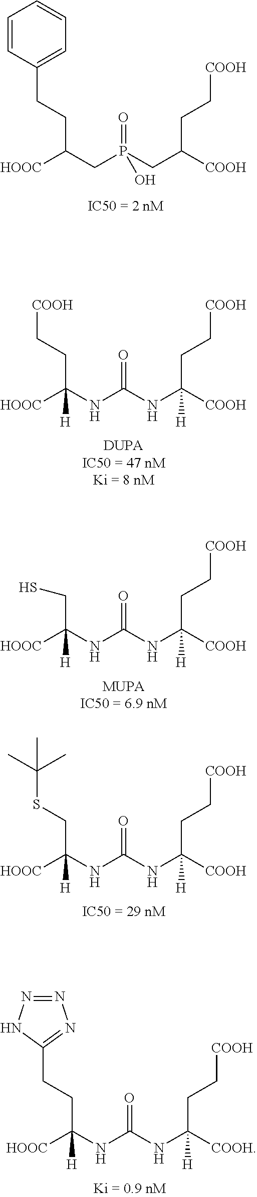 [SMILES: O=C(O)CCC(CP(=O)(O)CC(CCc1ccccc1)C(=O)O)C(=O)O ; O=C(O)CC[C@H](NC(=O)N[C@@H](CCC(=O)O)C(=O)O)C(=O)O ; O=C(O)CC[C@H](NC(=O)N[C@@H](CS)C(=O)O)C(=O)O ; CC(C)(C)SC[C@H](NC(=O)N[C@@H](CCC(=O)O)C(=O)O)C(=O)O ; O=C(O)CC[C@H](NC(=O)N[C@@H](CCc1nnn[nH]1)C(=O)O)C(=O)O]

In some embodiments, the PSMA bonding ligand is 2-[3-(1-Carboxy-2-mercapto-ethyl)-ureido]-pentanedioic acid (MUPA) or 2-[3-(1,3-Dicarboxy-propyl)-ureido]-pentanedioic acid (DUPA).

In any of the imaging agent compositions described herein, the targeting molecule can be the neutral compound or a pharmaceutically acceptable salt thereof.

### EXAMPLES

### Example 1

### Preparation of Compound II

Compound II was prepared according to the following scheme as taught in US patent publication number US20100324008 A1, which is incorporated herein by reference.

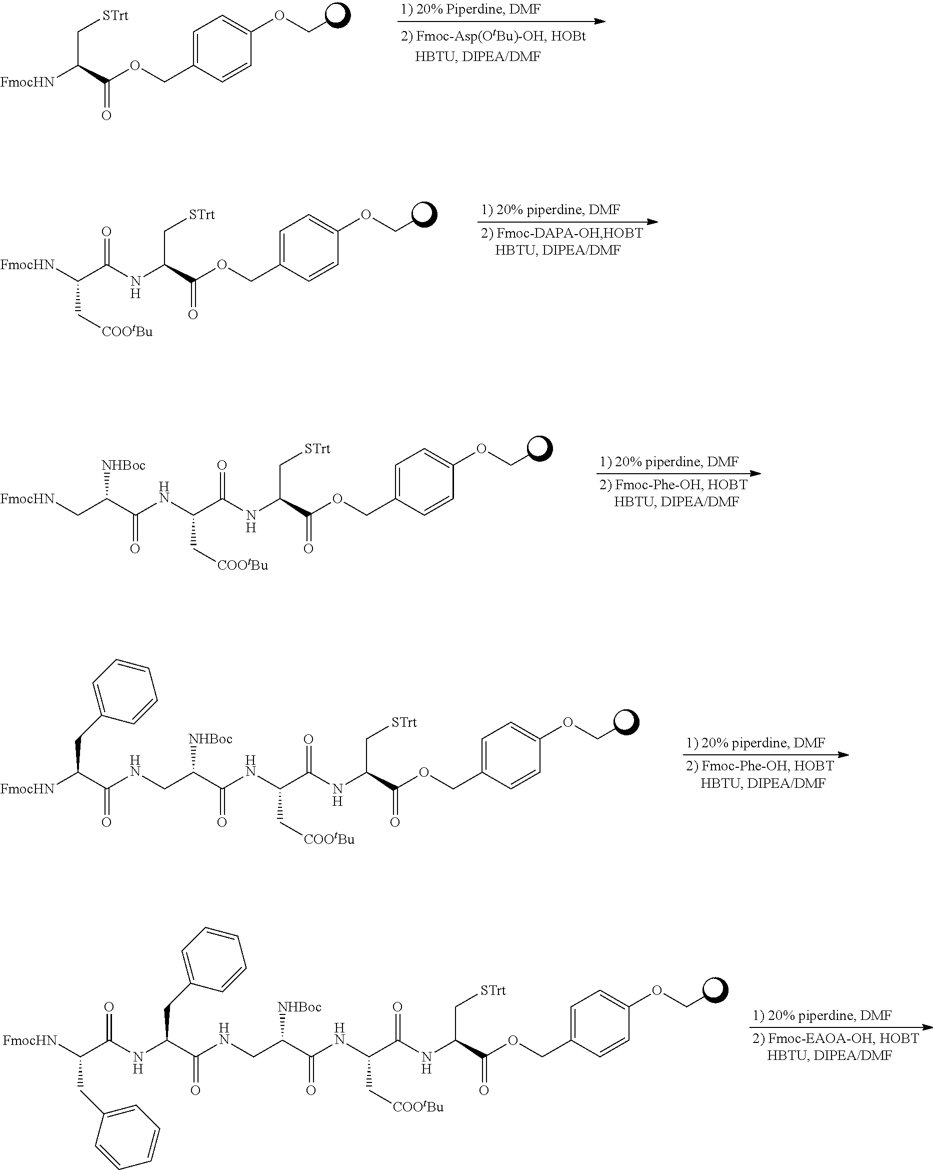

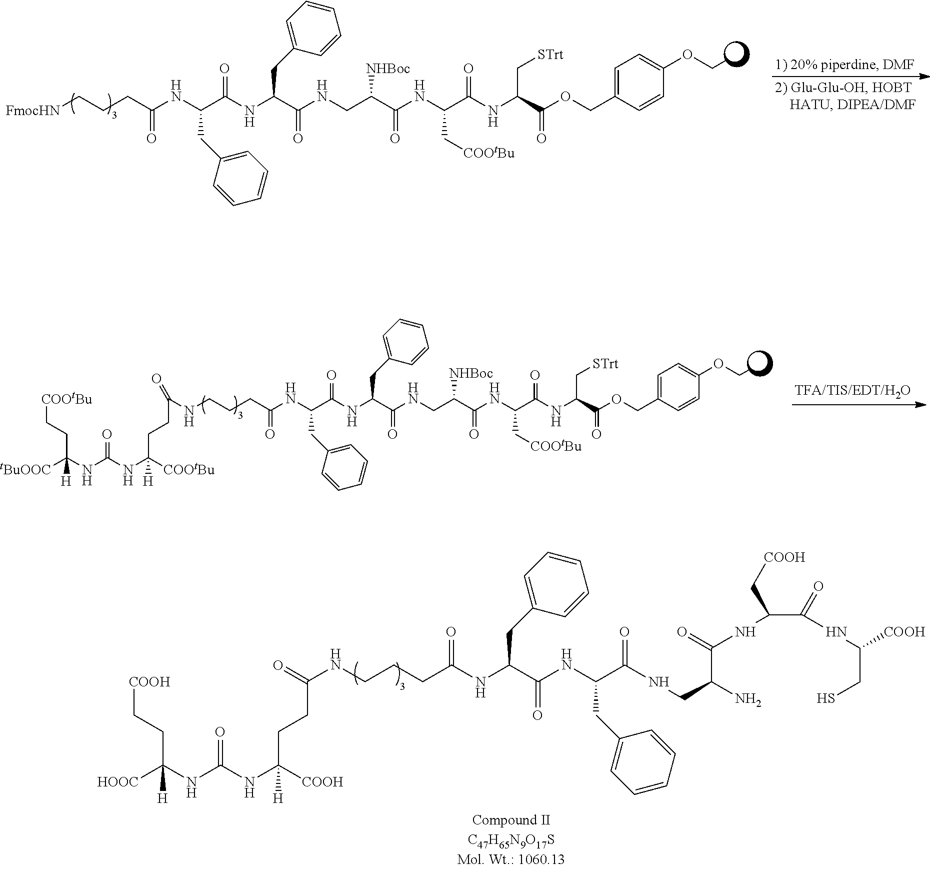

Compound II was synthesized using standard fluorenylmethyloxycarbonyl (Fmoc) solid phase peptide synthesis (SPPS) starting from Fmoc-Cys(Trt)-Wang resin (Novabiochem; Catalog #04-12-2050). Compound II was purified using reverse phase preparative HPLC (Waters, xTerra C₁₈ 10 μm; 19×250 mm) A=0.1 TFA, B=Acetonitrile (ACN); λ=257 nm; Solvent gradient: 5% B to 80% B in 25 min, 80% B wash 30 min run, (61%). Purified compounds were analyzed using reverse phase analytical HPLC (Waters, X-Bridge Bridge C₁₈ 5 μm; 3.0×15 mm); A=0.1 TFA, B=ACN; λ=257 nm, 5% B to 80% B in 10 min, 80% B wash 15 min run. C₄₇H₆₅N₂O₁₇S; MW=1060.13 g/mol; white solid; R₁=7.7 min; ¹H NMR (DMSO-d₆/D₂O) δ 0.93 (m, 2H); 1.08 (m, 5H); 1.27 (m, 5H); 1.69 (m, 2H); 1.90 (m, 2H); 1.94 (m, 2H); 2.10 (m, 2H); 2.24 (q, 2H); 2.62 (m, 2H); 2.78 (m, 4H); 2.88 (dd, 1H); 2.96 (t, 2H); 3.01 (dd, 1H); 3.31 (dd, 1H); 3.62 (dd, 1H); 3.80 (q, 1H, αH); 4.07 (m, 1H, αH); 4.37 (m, 1H, αH); 4.42 (m, 2H, αH); 4.66 (m, 1H, αH); 7.18 (m, 10H, Ar—H): LC-MS=1061 (M+H)₊; ESI-MS=1061 (M+H)⁺.

### Example 2

### Preparation of Compound II Formulation

A 12 liter volume of Water For Injection (WFI) was sparged with nitrogen. Solutions of 1.0 M NaOH and 0.2 M HCl were prepared and sparged with nitrogen for pH adjustment of the formulation and for preparation of the stannous chloride stock solution. 2000 mL of deoxygenated WFI was added to a 5L jacketed formulation vessel which was connected to a chiller. The chiller solution was set at 5° C. and circulation was maintained throughout the compounding and filtration process. 88.6 g of sodium gluconate and 1063 mg of EDTA disodium dihydrate were weighed and transferred to the formulation vessel and dissolved. A stannous chloride stock solution at a concentration of 10 mg/mL was made using the previously prepared 0.2 M HCl. A 35.4 mL aliquot of the stannous chloride stock solution was added to the formulation vessel and mixed well with stirring. 354.3 mg (net content) of Compound II was weighed and transferred into the formulation vessel. The mixture was stirred for at least 5 minutes and complete dissolution was observed. The pH was adjusted to 6.8±0.2 with deoxygenated 1.0 M NaOH solution and 0.2 N HCl solution. Deoxygenated WFI was then added until a formulation weight of 3578 g (3543 mL) was achieved. The formulation solution was stirred for five minutes and then sterile filtered through a 0.22 μm filter into a receiving vessel. Vials were filled with 1.01 g±0.03 g (1.00 mL) solution per vial. The vials were loaded into the lyophilizer. Inert atmosphere via a nitrogen blanket was maintained throughout formulation and vialing. Upon completion of the lyophilization cycle, vials were backfilled with nitrogen to approximately 646,000 mTorr. The vials were stoppered and removed from the lyophilizer, crimped with aluminum seals and labeled. Vials were placed in boxes and were stored at 5±3° C.

### Example 3

### Room Temperature Labeling of Compound II with ^{99m}Tc to Provide Imaging Agent of Formula I (^{99m}Tc-Compound II)

A Compound II kit vial from Example 2 was removed from the refrigerator and allowed to warm to room temperature (17-27° C.) for 15-30 min. The vial was put into a suitable radioactive shielding container. One to Two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then allowed to stand at ambient temperature (17-27° C.) for 15 minutes. 5-6 mL of 0.9% sodium chloride injection, USP, was then added to the vial. The labeled solution was stored at room temperature (17-27° C.) and used within 6 hours of preparation.

### Example 4

### Determination of Radiochemical Purity of ^{99m}Tc-Compound II by Radio-HPLC

The Radio-HPLC system used for the following experiment consisted of a Waters 600 intelligent pump, a Bioscan Flow-Count radiodetector, and a Waters Nova-Pak C18 (3.9×150 mm) column, using Laura v1.5 radiochromatogram software.

1-5 μL of the ^{99m}Tc-Compound II sample was injected into the HPLC and eluted with an aqueous mobile phase 0.1% trifluoroacetic acid in water (A) and Acetonitrile (B) at a linear gradient of 25% B to 35% B over 20 minutes at a flow rate of 1 mL/min. The ^{99m}Tc-Compound II showed two peaks which represent the expected pair of isomers. The radiochemical purity of ^{99m}Tc-Compound II was calculated as follows:

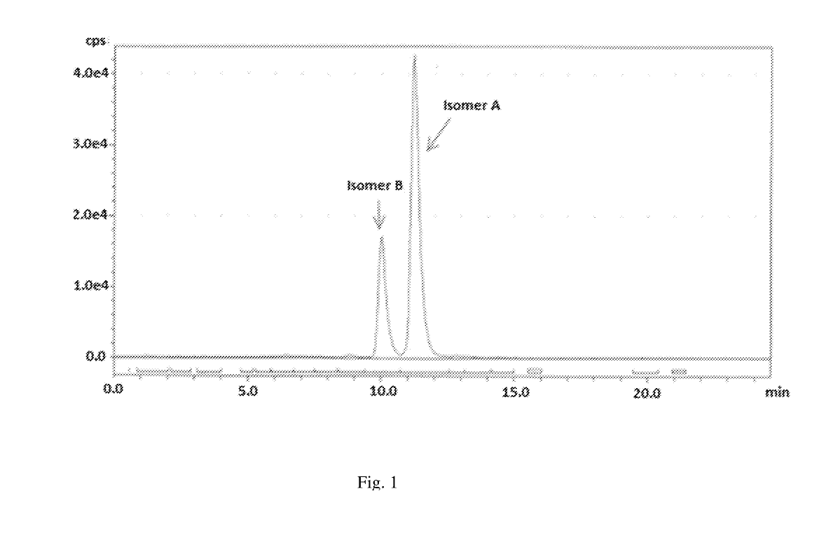

Radiochemical purity=isomer A %+isomer B % (FIG. 1)

| | |
|---|---|
| Channel: Pulse 1 | Detector: |

| RT | Height | Area | % ROI | |
|---|---|---|---|---|
| (m) | (cps) | (Counts) | (%) | |
| 1.2 | 291.9 | 5169.4 | 0.37 | |
| 2.4 | 82.0 | 1665.5 | 0.12 | |
| 3.4 | 131.3 | 3087.1 | 0.22 | |
| 5.0 | 86.6 | 1817.6 | 0.13 | |
| 5.5 | 107.7 | 2705.0 | 0.19 | |
| 6.3 | 337.3 | 10110.2 | 0.71 | |
| 6.9 | 239.4 | 7276.7 | 0.51 | |
| 7.7 | 131.1 | 3779.4 | 0.27 | |
| 8.8 | 413.2 | 10464.0 | 0.74 | |
| 10.0 | 17098.2 | 364139.4 | 25.75 | isomer B |
| 11.2 | 42824.2 | 986005.9 | 69.72 | isomer A |
| 12.8 | 385.5 | 9019.3 | 0.64 | |
| 13.2 | 238.8 | 5643.4 | 0.40 | |
| 14.3 | 87.0 | 2044.8 | 0.14 | |
| 15.8 | 31.4 | | | |
| 19.8 | 79.6 | 1351.4 | 0.10 | |
| 21.2 | 14.0 | | | |
| | | N/A | 100.00 | |

Total Area = 1418374.1 Counts
Bkg Area = 27472.8 Counts
Unallocated = 4094.8 Counts (0.29%)
99mTc-EC0652
Analyzed immediately after labeling
RCP = Isomer A % + Isomer B % = 25.75% + 69.72% = 95.5%

### Example 5

### Determination of Radiochemical Purity of ^{99m}Tc-Compound II by TLC

This TLC method determines the amount of each impurity using two systems:

System A: Instant Thin Layer Chromatography-Silica Gel (ITLC-SG) plate developed by saturated sodium chloride solution to detect free ^{99m}Tc-pertechnetate and ^{99m}Tc-gluconate/EDTA.

System B: ITLC-SG plate developed by 0.1% sodium dibasic phosphate solution to detect reduced-hydrolyzed colloidal ^{99m}Tc.

Method: Saturated sodium chloride solution and 0.1% sodium dibasic phosphate solution were each poured into separate developing tanks to a depth of about 0.5 cm. Two ITLC-SG plates were marked with a pencil at the edge at 1.5 cm (origin) and 6.5 cm (solvent front) from the bottom. Diagrams of the plates for System A and System B are shown below.

System A: A small drop (1 to 10 μL) of ^{99m}Tc-Compound II solution was applied to each ITLC-SG plate at the origin using a syringe and placed in the developing tank containing saturated sodium chloride solution upright against the side of the tank, so that the origin was above the solvent line. The developing tank was covered.

System B: One or two drops (10-20 μL) of ethanol were applied to an ITLC-SG plate at the origin and allowed to dry in air for about 30-60 seconds. A small drop (1 to 10 μL) of ^{99m}Tc- Compound II solution was then applied on the ethanol spot and immediately placed in the developing tank containing 0.1% sodium dibasic phosphate solution upright against the side of the tank, so that the origin was above the solvent line. The developing tank was covered.

The plates were removed from both tanks after the solvent front migrated 5.0 cm from the origin of each plate.

The plate developed by saturated sodium chloride solution was cut into two pieces at 3.0 cm from origin and counted using appropriate counting equipment. The percent of ^{99m}Tc pertechnetate and ^{99m}Tc-gluconate/EDTA is calculated as follows:

A=% Pertechnetate and ^{99m}Tc-gluconate/EDTA=(activity in top piece/activity in both pieces)×100

The plate developed by 0.1% sodium dibasic phosphate solution was cut into two pieces at 1 cm from the origin and counted. The percent of reduced-hydrolyzed ^{99m}Tc is calculated as follows:

B=% reduced-hydrolyzed ^{99m}Tc=activity in bottom piece/activity in both pieces)×100

The radiochemical purity was calculated as 100−(A+B).

### Comparative Example 1

### Preparation of Compound II Prior Art Formulation

An 11 liter volume of Water For Injection (WFI) was sparged with nitrogen. Solutions of 1.0 M NaOH and 0.2 M HCl were prepared and sparged with nitrogen for pH adjustment of the formulation and for preparation of the stannous chloride stock solution. 1050 mL of deoxygenated WFI was added to a 5 L media bottle. 84 grams of sodium glucoheptonate dihydrate was weighed and transferred to the formulation vessel and dissolved. A stannous chloride stock solution at a concentration of 10 mg/mL was made using the previously prepared 0.2 M HCl. A 8.4 mL aliquot of the stannous chloride stock solution was added to the formulation vessel and mixed well with stirring. 150 mg (net content) of Compound II was weighed and transferred into the formulation vessel. The mixture was stirred for at least 5 minutes and complete dissolution was observed. The pH was adjusted to 6.8±0.2 with deoxygenated 1.0 M NaOH solution and 0.2 N HCl solution. Deoxygenated WFI was then added until a formulation weight of 1545 g (1500 mL) was achieved. The formulation solution was stirred for five minutes and then sterile filtered through a 0.22 μm filter into a receiving vessel. Vials were filled with 1.03 g±0.03 g (1.00 mL) solution per vial. The vials were loaded into the lyophilizer. Inert atmosphere via nitrogen blanket was maintained throughout formulation and vialing. Upon completion of the lyophilization cycle, vials were backfilled with nitrogen to approximately 646,000 mTorr. The vials were stoppered and removed from the lyophilizer, crimped with aluminum seals and labeled. Vials were placed in boxes and were stored at 5±3° C.

### Comparative Example 2

### Prior Art Method of Labeling Compound II with ^{99m}Tc to Provide Imaging Agent of Formula I (^{99m}Tc-Compound II)

A Compound II kit vial from Comparative Example 1 was removed from the refrigerator and allowed to warm to room temperature (17-27° C.) for 15-30 min. The vial was put into a suitable radioactive shielding container. One to two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then heated in a heating bloc at 100° C. or boiling water bath for 10 minutes. After cooling to room temperature for 10-15 minutes, 5-6 mL of 0.9% sodium chloride injection, USP, was then added. The labeled solution was stored at room temperature (17-27° C.) and used within 6 hours of preparation.

### Direct Comparison Example 1

### Direct Comparison of Cold Labelling of Inventive Compound II Formulation with Prior Art Compound II Formulation

Using the methods described herein, kit formulations DC1A (prior art comparative example) and DC1B (described herein) of Compound II were prepared as shown in Table 1.

TABLE 1

| | | | | Radiochemical |
|---|---|---|---|---|
| | | | | Purity of ^{99m}Tc- |
| Example | Kit Formulation | | Labeling Condition | Compound II |
| DC1A | 0.1 mg Compound II | 1. | Added 33 mCi (1.0 mL) of | 84% |
| | 0.056 mg Tin chloride dihydrate | | ^{99m}Tc pertechnetate to a kit vial. | |
| | 56 mg sodium glucoheptonate | 2. | Incubated at room temperature | |
| | dihydrate | | (22° C.) for 15 min. | |
| | pH 6.8 | | | |
| DC1B | 0.1 mg Compound II | 1. | Added 33 mCi (1.0 mL) of | 98% |
| | 0.1 mg Tin chloride dihydrate | | ^{99m}Tc pertechnetate to a kit vial. | |
| | 25 mg sodium gluconate | 2. | Incubated at room temperature | |
| | 0.3 mg EDTA disodium | | (22° C.) for 15 min. | |
| | dihydrate | | | |
| | pH 6.8 | | | |

Room Temperature ^{99m}Tc Labelling: A Compound II kit vial (kit vial 3A or 3B) was removed from the refrigerator and allowed to warm to room temperature for 15-30 min. The vial was put into a suitable radioactive shielding container. One to Two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then allowed the vial to stand at ambient temperature (17-27° C.) for 15 minutes. 5-6 mL of 0.9% sodium chloride injection, USP, was then added to the vial.

Comparison of radiochemical purities:

The radiochemical purity of ^{99m}Tc-Compound II from Example 3A and 3B was determined by HPLC as described herein.

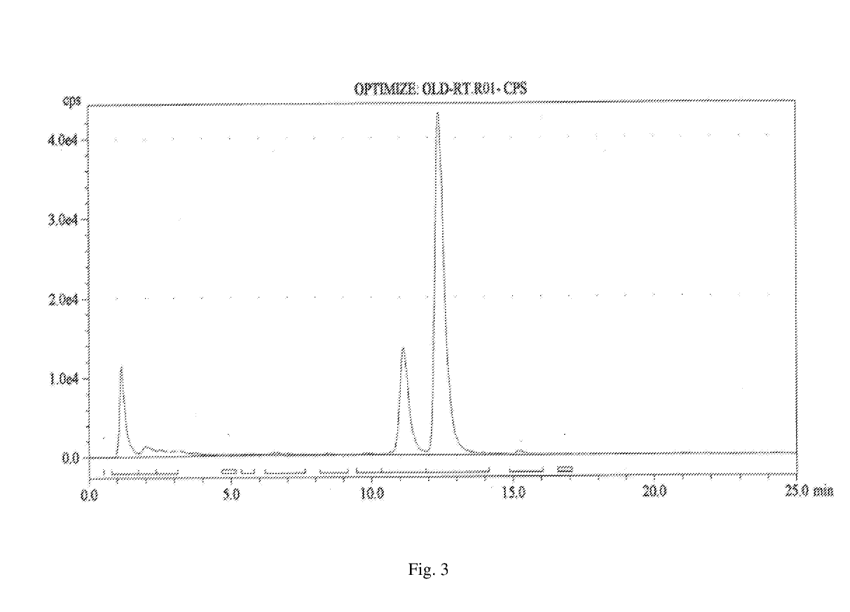

The radiochemical purity of ^{99m}Tc-Compound II prepared by reconstituting an Example DC1A kit vial and incubating at room temperature was 84% (FIG. 3).

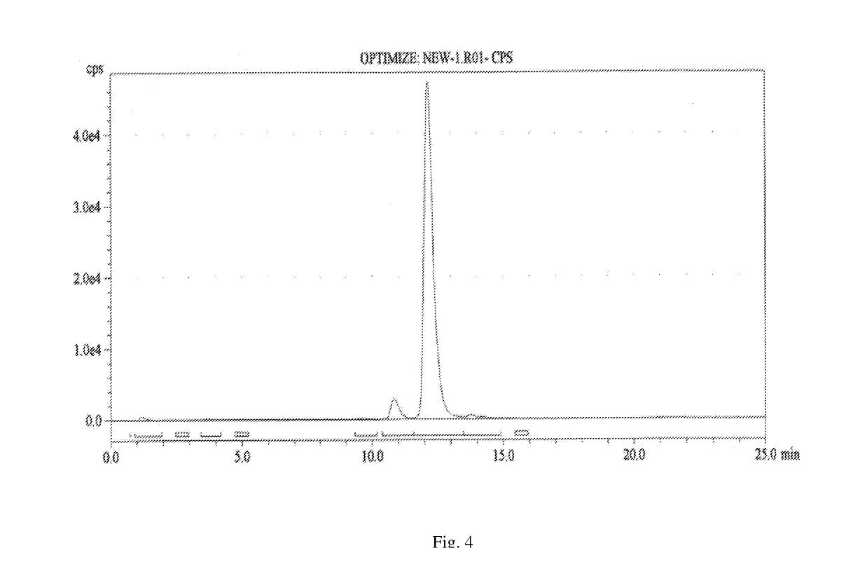

The radiochemical purity of ^{99m}Tc-Compound II prepared by reconstituting an Example DC1B kit vial and incubating at room temperature was 98% (FIG. 4).

### Example 6

### Preparation of Compound IV

Compound IV was prepared according to the following scheme as taught in U.S. Pat. No. 7,128,893, which is incorporated herein by reference.

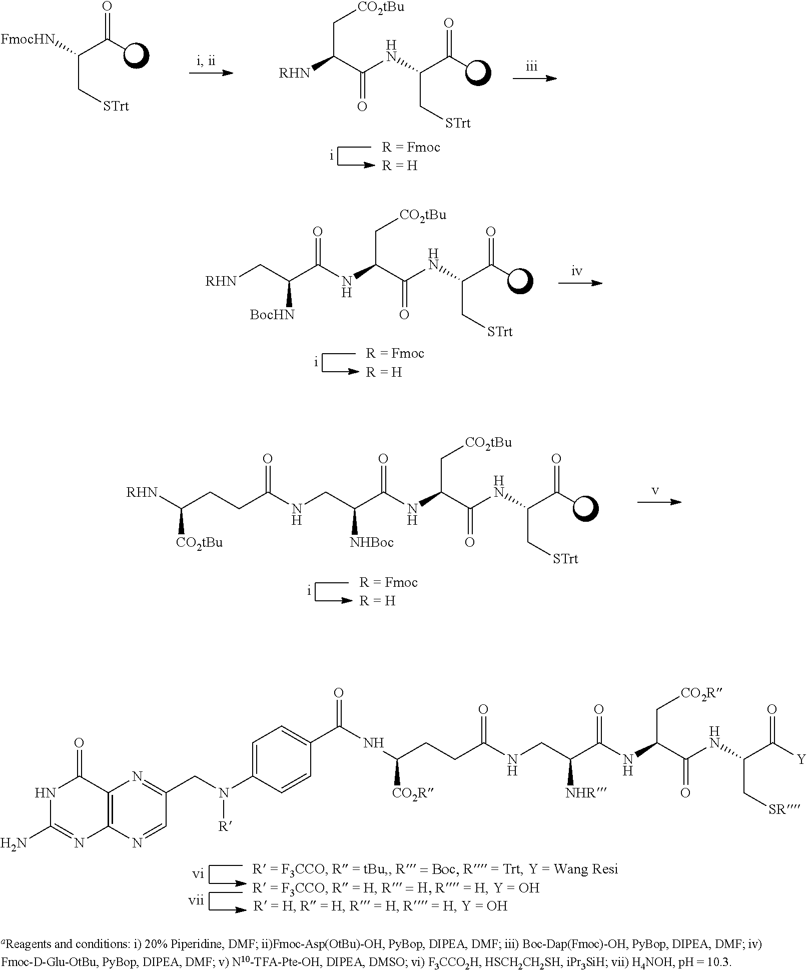

EC20 was prepared by a polymer-supported sequential approach using the Fmoc-strategy (see Scheme 1 below; Fmoc=9-fluorenylmethyloxycarbonyl; Boc=tert-butyl-oxycarbonyl; Dap=diaminopropionic acid; DMF=dimethylformamide; DIPEA=diisopropyl-ethylamine). EC20 was synthesized on an acid-sensitive Wang resin loaded with Fmoc-_{L}-Cys(Trt)-OH. Benzotriazole-1-yl-oxy-tris-pyrrolidino-phosphonium-hexafluorophosphate (PyBOP) was applied as the activating reagent to ensure efficient coupling using low equivalents of amino acids. Fmoc protecting groups were removed after every coupling step under standard conditions (20% piperidine in DMF). After the last assembly step the peptide was cleaved from the polymeric support by treatment with 92.5% trifluoroacetic acid containing 2.5% ethanedithiol, 2.5% triisopropylsilane and 2.5% deionized water. This reaction also resulted in simultaneous removal of the t-Bu, Boc and trityl protecting groups. Finally, the trifluoroacetyl moiety was removed in aqueous ammonium hydroxide to give EC20.

### Example 7

### Preparation of Inventive EC20 Formulation Kit

A 2 liter volume of Water For Injection (WFI) was sparged with nitrogen. Solutions of 1.0 M NaOH and 0.2 M HCl were prepared and sparged with nitrogen. These solutions are used for pH adjustment of the formulation and for preparation of the stannous chloride stock solution. 500 mL of deoxygenated WFI was added to a 2 L jacketed formulation vessel, which was connected to a chiller. The chiller solution was set at 5° C. and circulation was maintained throughout the compounding and filtration process. 25.0 g of sodium gluconate and 300 mg of EDTA disodium dihydrate were weighed and transferred to the formulation vessel. The mixture was stirred until all of the solids had dissolved. A stannous chloride stock solution at a concentration of 10 mg/mL was made using the previously prepared 0.2 M HCl. A 10.0 mL (100 mg of SnCl₂.2H₂O) aliquot of the stannous chloride stock solution was added to the formulation vessel and mixed well with stirring. 100 mg (net content) of EC20 was weighed and transferred into the formulation vessel. The mixture was stirred for at least 5 minutes and complete dissolution was observed. The pH was adjusted to 6.8±0.2 with deoxygenated 1.0 M NaOH solution and 0.2 N HCl solution. Deoxygenated WFI was then added until a formulation weight of 1010 g (1000 mL) was achieved. The formulation solution was stirred for five minutes and then sterile filtered through a 0.22 μm filter into a receiving vessel. Vials were filled with 1.01 g±0.05 g (1.00 mL) solution per vial. The vials were loaded into a lyophilizer. Full inerting using a nitrogen blanket was maintained throughout formulation and vialing. Upon completion of the lyophilization cycle, vials were backfilled with nitrogen. The vials were stoppered and removed from the lyophilizer, crimped with aluminum seals and labeled. Vials were stored at 5±3° C.

### Example 8

### Labeling EC20 with ^{99m}Tc using Inventive Formulation Fit

An EC20 kit vial (prepared in Example 7) was removed from the refrigerator and allowed to warm to room temperature for 15-30 min. The vial was put into a suitable radioactive shielding container. One to two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then allowed to stand at ambient temperature (22±5° C.) for 15 minutes. The labeled solution was stored at room temperature and used within 6 hours of preparation.

### Comparative Example 3

### Preparation of EC20 Prior Art Formulation Kit

A 5 liter volume of Water For Injection (WFI) was sparged with nitrogen. Solutions of 1.0 M NaOH and 0.2 M HCl were prepared and sparged with nitrogen for pH adjustment of the formulation and for preparation of the stannous chloride stock solution. Sodium glucoheptonate stock solution (0.1667 g/mL) was prepared by dissolving 500 g of sodium glucoheptonate dihydrate in 3000 mL of deoxygenated WFI and filtering through a 0.22 μm sterile filter. A stannous chloride stock solution at a concentration of 10 mg/mL was made using the previously prepared 0.2 M HCl. A bulk solution of excipients was prepared by mixing 2875 mL of sodium glucoheptonate stock solution (479 g of sodium glucoheptonate) and 48 mL of stannous chloride stock solution (480 mg of stannous chloride), adjusting the pH to 6.8±0.2 with 1.0 M NaOH and 0.2 M HCl and diluting to 6000 mL with WFI. The EC20 formulation solution was prepared by dissolving 4856 mg (net content) of EC20 drug substance in 4856 mL of the excipients solution (pH 6.8±0.2). The formulation solution was then sterile filtered through a 0.22 μm filter into a receiving vessel. Vials were filled with 1.03 g±0.05 g (1.00 mL) solution per vial. The vials were loaded into the lyophilizer. Full inerting using a nitrogen blanket was maintained throughout formulation and vialing. Upon completion of the lyophilization cycle, vials were backfilled with nitrogen. The vials were stoppered and removed from the lyophilizer, crimped with aluminum seals and labeled. Vials were stored at 5±3° C.

### Comparative Example 4

### Prior Art Method of Labeling Compound IV with ^{99m}Tc to Provide Imaging Agent of Formula III (^{99m}Tc-Compound IV)

An EC20 kit vial was removed from the refrigerator and allowed to warm to room temperature for 15-30 min. The vial was put into a suitable radioactive shielding container. One to two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then heated in a heating block at 100° C. or boiling water bath for 10 minutes. After heating, the vial was placed into a shielded container and cooled to room temperature for 10-15 minutes. The labeled solution was stored at room temperature and used within 6 hours of preparation.

### Example 9

### Determination of Radiochemical Purity of ^{99m}Tc-Compound IV by Radio-HPLC

The Radio-HPLC system consists of a waters alliance HPLC system, a Bioscan Flow-Count radiodetector and a Waters Sunfire C18 (3.0×100 mm) column. 1-10 μL of the ^{99m}Tc-EC20 sample were injected into the HPLC and eluted with an aqueous mobile phase 0.1% trifluoroacetic acid in water (A) and methanol (B) at a linear gradient of 20% B to 45% B in 20 minutes at a flow rate of 0.5 mL/min. The ^{99m}Tc-EC20 shows two peaks (FIG. 1) which are a pair of isomers. The radiochemical purity of ^{99m}Tc-EC20 is calculated as follow:

Radiochemical purity=isomer A %+Isomer B %.

### Direct Comparison Example 2

### Direct Comparison of Cold Labelling of Inventive Compound IV Formulation with Prior Art Compound IV Formulation

Using the methods described herein, kit formulations 6A (prior art comparative example) and 6B (described herein) of Compound IV were prepared as shown in Table 2.

TABLE 2

| | | | | | Radiochemical |
|---|---|---|---|---|---|
| | Kit Formulation | | Labeling Conditions | | Purity |
| 6A | (1) | 0.1 mg EC20 | (1) | Added 40 mCi of | 82.5% |
| | (2) | 80 mg sodium | | ^{99m}Tc pertechnetate | |
| | | glucoheptonate | | to a kit. | |
| | | dihydrate | (2) | Incubated at room | |
| | (3) | 0.08 mg tin chloride | | temperature (22° C.) | |
| | (4) | dihydrate | | for 20 min. | |
| | | pH 6.8 | | | |
| 6B | (1) | 0.1 mg EC20 | (1) | Added 40 mCi of | 98.2% |
| | (2) | 25 mg sodium | | ^{99m}Tc pertechnetate | |
| | | gluconate | | to a kit. | |
| | (3) | 0.3 mg EDTA | (2) | Incubated at room | |
| | (4) | 0.1 mg tin (II) | | temperature (22° C.) | |
| | | chloride dihydrate | | for 15 min. | |
| | (5) | pH 6.8 | | | |

Room Temperature ^{99m}Tc Labelling: A Compound IV kit vial (kit vial 6A or 6B) was removed from the refrigerator and allowed to warm to room temperature for 15-30 min. The vial was put into a suitable radioactive shielding container. One to Two milliliter (≦50 mCi) of ^{99m}Tc pertechnetate injection was added to the vial using a lead shielded syringe. Before removing the syringe from the vial, equal volume of headspace was withdrawn in order to normalize the pressure inside the vial. The vial was gently swirled to completely dissolve the powder and then allowed the vial to stand at ambient temperature (17-27° C.) for 15 minutes. 5-6 mL of 0.9% sodium chloride injection, USP, was then added to the vial.

Comparison of radiochemical purities:

The radiochemical purity of ^{99m}Tc-Compound IV from Example 6A and 6B was determined by HPLC as described herein.

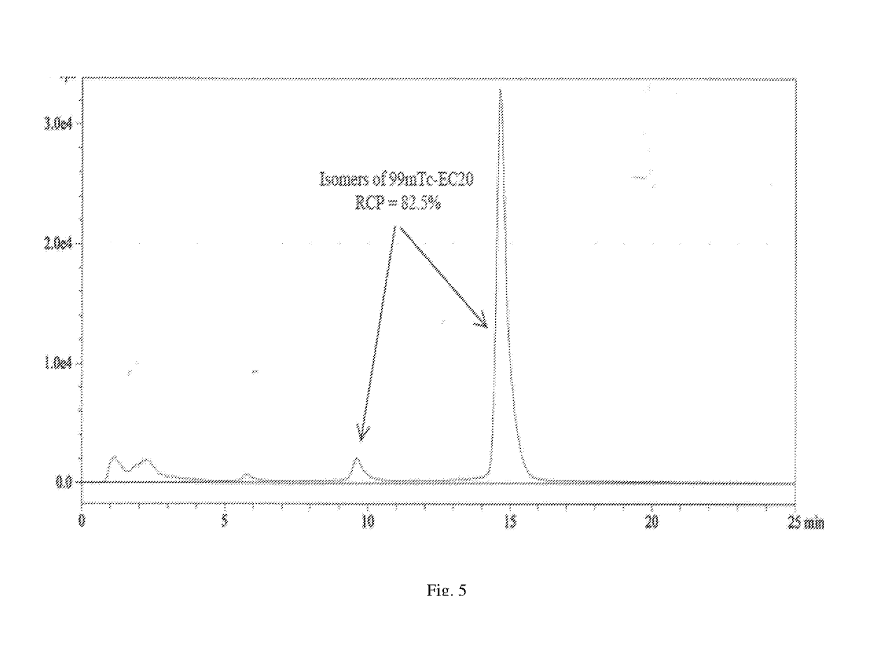

The radiochemical purity of ^{99m}Tc-Compound IV prepared by reconstituting an Example 6A kit vial and incubating at room temperature was 82.5% (FIG. 5).

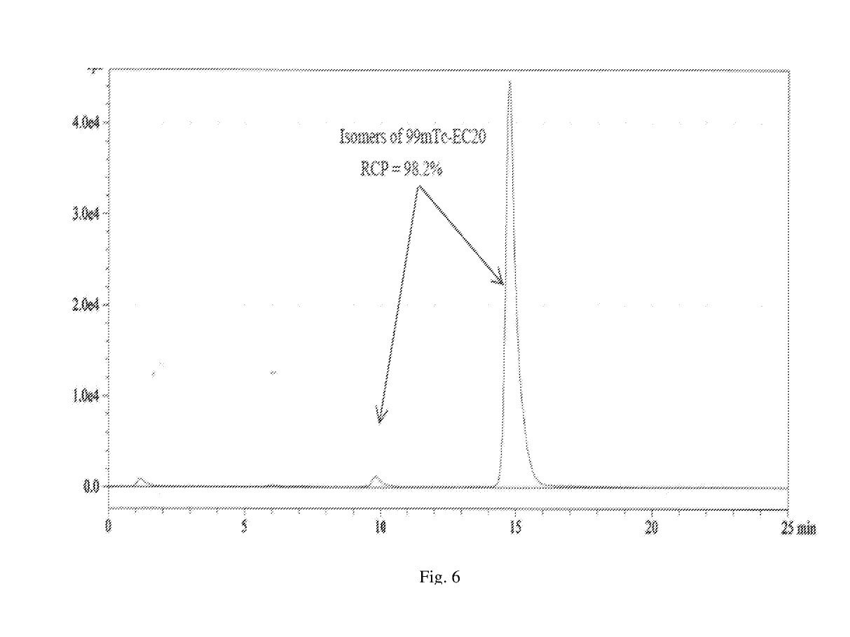

The radiochemical purity of ^{99m}Tc-Compound IV prepared by reconstituting an Example 6B kit vial and incubating at room temperature was 98.2% (FIG. 6).

### Example 10

### Effect of pH on Labelling and Stability of Compound II

Using the methods described herein, Compound II was subjected to room temperature labelling at varying pH. Results are shown in Table 3.

TABLE 3

| Kit Formulation | | | | | Radiochemical | |
|---|---|---|---|---|---|---|
| | Sodium | | Tin | | Purity (%) | |
| EC0652 | Gluconate | EDTA | Chloride | | Time | Time |
| (mg) | (mg) | (mg) | (mg) | pH | (0 Hr) | (6 Hr) |
| 0.1 | 25 | 0.3 | 0.1 | 5.1 | 97.7 | 85.5 |
| 0.1 | 25 | 0.3 | 0.1 | 6.2 | 96.8 | 94.1 |
| 0.1 | 25 | 0.3 | 0.1 | 6.8 | 97.4 | 94.8 |
| 0.1 | 25 | 0.3 | 0.1 | 7.4 | 96.5 | 96.0 |

### Example 11

### Effect of Tin Concentration on Labelling of Compound II

Using the methods described herein, Compound II was subjected to room temperature labelling with varying amounts of tin chloride. Results are shown in Table 4.

TABLE 4

| Kit Formulation | | | | | |
|---|---|---|---|---|---|
| | Sodium | | Tin | | Radiochemical |
| EC0652 | Gluconate | EDTA | Chloride | | Purity (%) |
| (mg) | (mg) | (mg) | (mg) | pH | Time (0 Hr) |
| 0.10 | 25 | 0.3 | 0.01 | 6.8 | 72.7 |
| 0.10 | 25 | 0.3 | 0.02 | 6.8 | 95.4 |
| 0.10 | 25 | 0.3 | 0.04 | 6.8 | 96.8 |
| 0.10 | 25 | 0.3 | 0.10 | 6.8 | 97.0 |
| 0.10 | 25 | 0.3 | 0.15 | 6.8 | 97.0 |
| 0.10 | 25 | 0.3 | 0.30 | 6.8 | 93.1 |

### Example 12

### Effect of EDTA on Labelling and Stability of Compound II

Using the methods described herein, Compound II was subjected to room temperature labelling with varying amounts of EDTA. Results are shown in Table 5.

TABLE 5

| Kit Formulation | | | | | | |
|---|---|---|---|---|---|---|
| | Sodium | | Tin | | RCP (%) | |
| EC0652 | Gluconate | EDTA | Chloride | | Time | Time |
| (mg) | (mg) | (mg) | (mg) | pH | (0 Hr) | (6 Hr) |
| 0.1 | 25 | 0 | 0.1 | 6.8 | 85.37 | 88.06 |
| 0.1 | 25 | 0.005 | 0.1 | 6.8 | 83.69 | 74.46 |
| 0.1 | 25 | 0.01 | 0.1 | 6.8 | 85.92 | 94.03 |
| 0.1 | 25 | 0.1 | 0.1 | 6.8 | 95.77 | 95.33 |
| 0.1 | 25 | 0.30 | 0.1 | 6.8 | 97.02 | 97.49 |
| 0.1 | 25 | 1.0 | 0.1 | 6.8 | 96.69 | 96.87 |

### Example 13

### Effect of Sodium Gluconate on Labelling and Stability of Compound II

Using the methods described herein, Compound II was subjected to room temperature labelling with varying amounts of sodium gluconate. Results are shown in Table 6.

TABLE 6

| Kit Formulation | | | | | Colloid | |
|---|---|---|---|---|---|---|
| | Sodium | | Tin | | ^{99m}Tc (%) | RCP (%) |
| EC0652 | Gluconate | EDTA | Chloride | | Time | Time |
| (mg) | (mg) | (mg) | (mg) | pH | (0 Hr) | (6 Hr) |
| 0.1 | 1 | 0.3 | 0.1 | 6.8 | 1.23 | 91.7 |
| 0.1 | 20 | 0.3 | 0.1 | 6.8 | 0.34 | 97.9 |
| 0.1 | 25 | 0.3 | 0.1 | 6.8 | 0.55 | 98.5 |
| 0.1 | 30 | 0.3 | 0.1 | 6.8 | 0.36 | 97.0 |
| 0.1 | 50 | 0.3 | 0.1 | 6.8 | 0.36 | 98.0 |

1. An imaging agent composition comprising a targeting molecule, a chelating agent and a reducing agent, wherein the targeting molecule is of the formula 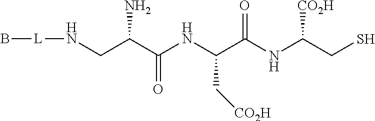
or a pharmaceutically acceptable salt thereof, wherein B is a binding ligand and L is an optional linker.

2. The imaging agent composition of claim 1, wherein B is a folate or a PSMA binding ligand.

3. (canceled)

4. The imaging agent composition of claim, wherein the optional linker L comprises at least two amino acid residues.

5. The imaging agent composition of claim 1, wherein the at least one chelating agent is selected from the group consisting of ethylene diamine tetraacetic acid, disodium ethylene diamine tetraacetic acid dihydrate, gluconic acid, lactic acid, citric acid, sodium gluconate, sodium lactate, sodium citrate, potassium gluconate, potassium lactate and potassium citrate.

6. The imaging agent composition of claim 1, wherein the wherein the chelating agent is a combination of sodium gluconate and disodium ethylene diamine tetraacetic acid dihydrate.

7. The imaging agent composition of claim 1, wherein the chelating agent is a combination of sodium gluconate and disodium ethylene diamine tetraacetic acid dihydrate in a ratio of about 25:1 to about 100:1 by weight.

8. The imaging agent composition of claim 1, wherein the reducing agent is stannous chloride.

9. The imaging agent composition of claim 1, having a pH in the range of about 6.5 to about 7.5.

10. (canceled)

11. (canceled)

12. The imaging agent composition of claim, further comprising a radiolabel source, wherein the radiolabel source is ^{99m}Tc-pertechneate.

13. (canceled)

14. The imaging agent composition of claim 12, wherein the targeting molecule and the radiolabel source combine to form an imaging agent of the formula 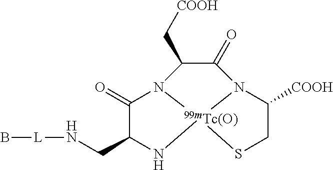
or a pharmaceutically acceptable salt thereof, wherein B is a binding ligand and L is an optional linker.

15. The imaging agent composition of claim 14, wherein the ^{99m}Tc-pertechnetate is in an amount in the range of about 1 mCi/mg to about 100 mCi/mg.

16. (canceled)

17. The imaging agent composition of claim 1, wherein the targeting molecule comprises a compound of the formula 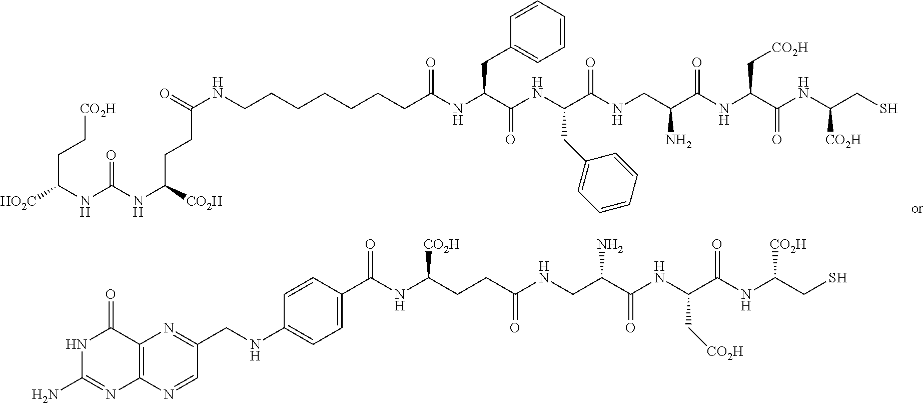
or a pharmaceutically acceptable salt thereof.

18.-23. (canceled)

24. A lyophilized imaging agent composition comprising a targeting molecule, two or more chelating agents selected from the group consisting of ethylene diamine tetraacetic acid, disodium ethylene diamine tetraacetic acid dihydrate, gluconic acid, lactic acid, citric acid, sodium gluconate, sodium lactate, sodium citrate, potassium gluconate, potassium lactate and potassium citrate, and a reducing agent, wherein the targeting molecule comprises a compound of the formula 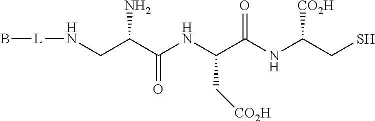
or a pharmaceutically acceptable salt thereof, wherein B is a binding ligand and L is an optional linker, and wherein the reducing agent is stannous chloride.

25. The lyophilized imaging agent composition of claim 24, wherein B is a folate or a PSMA binding ligand.

26. (canceled)

27. (canceled)

28. The lyophilized imaging agent composition of claim 24, wherein the two or more chelating agents are disodium ethylene diamine tetraacetic acid dihydrate and sodium gluconate.

29. The lyophilized imaging agent composition of claim 28, wherein the disodium ethylene diamine tetraacetic acid dihydrate and the sodium gluconate are in a ratio of about 25:1 to about 100:1 by weight.

30. The lyophilized imaging agent composition of claim 24, wherein the targeting molecule comprises a compound of the formula 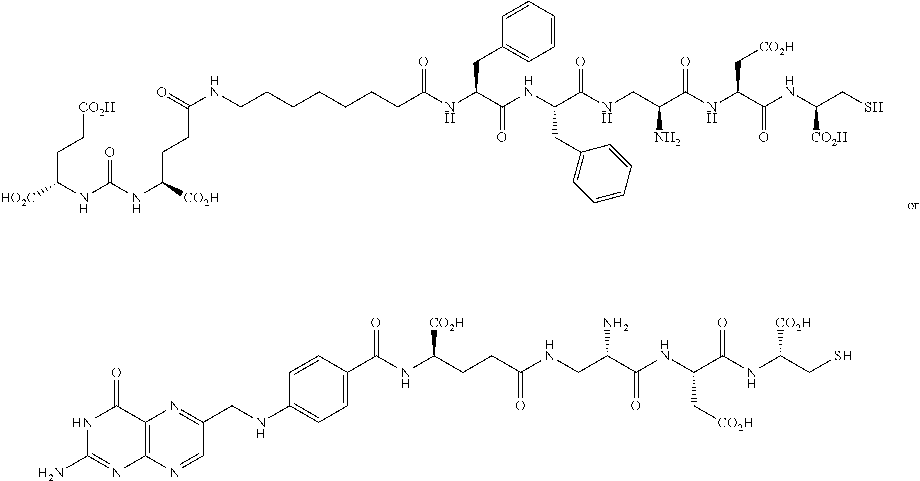
or a pharmaceutically acceptable salt thereof, and wherein the reducing agent is stannous chloride.

31.-37. (canceled)

38. A method for preparing an imaging agent composition comprising the steps of
(a) preparing a first solution comprising aqueous stannous chloride;
(b) preparing a second solution comprising aqueous stannous chloride, sodium gluconate and disodium ethylene diamine tetraacetic acid dihydrate by contacting the first solution with sodium gluconate and disodium ethylene diamine tetraacetic acid dihydrate in a vessel to form the second solution;
(c) preparing a third solution comprising aqueous stannous chloride, sodium gluconate disodium ethylene diamine tetraacetic acid dihydrate, and a compound of the formula 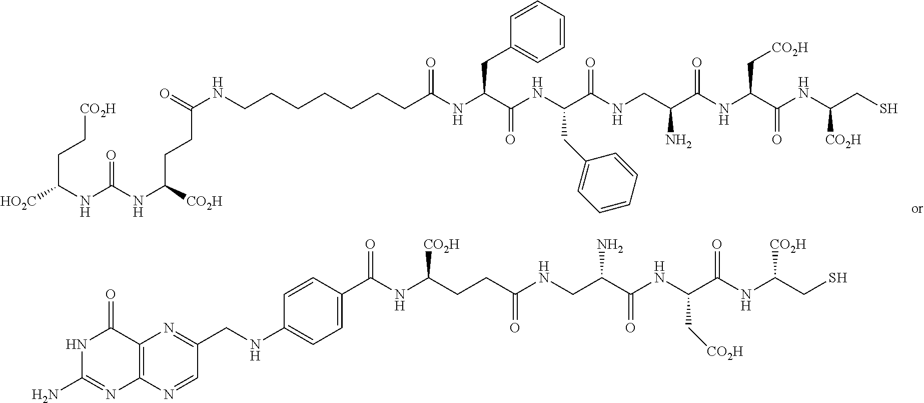
or a pharmaceutically acceptable salt thereof, by contacting the second solution with the compound of the formula 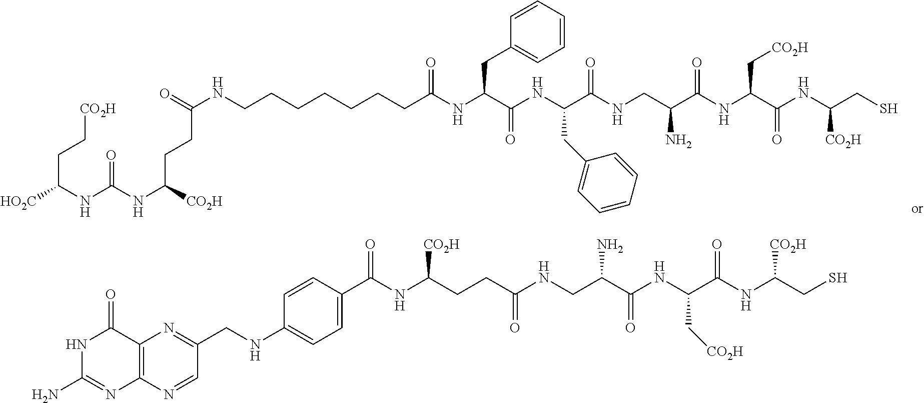
or a pharmaceutically acceptable salt thereof;
(d) adjusting the pH of the third solution to a pH in the range of about 6.5 to about 7.5; and
(e) lyophilizing the third solution form a lyophilized imaging agent composition.

39. The method of claim 38, further comprising the step of contacting the lyophilized imaging agent composition with an aqueous solution of ^{99m}Tc-pertechnetate.

40. The method of claim 39, wherein the step of contacting the lyophilized imaging agent composition with an aqueous solution of ^{99m}Tc-pertechnetate is conducted at a temperature of about 17° C. to about 27° C.

41.-43. (canceled)
